SEQUENCE LISTING (111 sequences)

SEQ ID NO: 1 - protein, 12 aa, Artificial Sequence
  Hair-binding Peptide
RTNAADHPAAVT

SEQ ID NO: 2 - protein, 7 aa, Artificial Sequence
  Hair-binding Peptide
DLTLPFH

SEQ ID NO: 3 - protein, 20 aa, Artificial Sequence
  Hair-binding peptide
THSTHNHGSPRHTNADAGNP

SEQ ID NO: 4 - protein, 11 aa, Artificial Sequence
  Hair-binding peptide
LPRIANTWSPS

SEQ ID NO: 5 - protein, 12 aa, Artificial Sequence
  Hair-Binding Peptide
EQISGSLVAAPW

SEQ ID NO: 6 - protein, 12 aa, Artificial Sequence
  Hair-Binding Peptide
TDMQAPTKSYSN

SEQ ID NO: 7 - protein, 12 aa, Artificial Sequence
  Hair-Binding Peptide
LDTSFPPVPFHA

SEQ ID NO: 8 - protein, 12 aa, Artificial Sequence
  Skin-binding Peptide
TPFHSPENAPGS

SEQ ID NO: 9 - protein, 7 aa, Artificial Sequence
  Skin-binding peptide
FTQSLPR

SEQ ID NO: 10 - protein, 12 aa, Artificial Sequence
  Skin-binding peptide
KQATFPPNPTAY

SEQ ID NO: 11 - protein, 12 aa, Artificial Sequence
  Skin-binding peptide
HGHMVSTSQLSI

SEQ ID NO: 12 - protein, 7 aa, Artificial Sequence
  Skin-binding peptide
LSPSRMK

SEQ ID NO: 13 - protein, 12 aa, Artificial Sequence
  Fingernail Binding-peptide
ALPRIANTWSPS

SEQ ID NO: 14 - protein, 12 aa, Artificial Sequence
  Fingernail-binding peptide
YPSFSPTYRPAF

SEQ ID NO: 15 - protein, 12 aa, Artificial Sequence
  Hair-Binding Peptide
TPPTNVLMLATK

SEQ ID NO: 16 - protein, 12 aa, Artificial Sequence
  Hair-binding Peptide
TPPELLHGDPRS

SEQ ID NO: 17 - protein, 12 aa, Artificial Sequence
  Hair-binding peptide
TPPELLHGAPRS

SEQ ID NO: 18 - protein, 7 aa, Artificial Sequence
  Hair-binding Peptide
NTSQLST

SEQ ID NO: 19 - protein, 15 aa, Artificial Sequence
  Hair-Binding Peptide
STLHKYKSQDPTPHH

SEQ ID NO: 20 - protein, 12 aa, Artificial Sequence
  Hair-Binding Peptide
GMPAMHWIHPFA

SEQ ID NO: 21 - protein, 15 aa, Artificial Sequence
  Hair-Binding Peptide
HDHKNQKETHQRHAA

SEQ ID NO: 22 - protein, 20 aa, Artificial Sequence
  Hair-Binding Peptide
HNHMQERYTDPQHSPSVNGL

SEQ ID NO: 23 - protein, 20 aa, Artificial Sequence
  Hair-Binding Peptide
TAEIQSSKNPNPHPQRSWTN

SEQ ID NO: 24 - protein, 12 aa, Artificial Sequence
  Skin-Binding Peptide
SVSVGMKPSPRP

SEQ ID NO: 25 - protein, 12 aa, Artificial Sequence
  Skin-Binding Peptide
TMGFTAPRFPHY

SEQ ID NO: 26 - protein, 12 aa, Artificial Sequence
  Skin-Binding Peptide
NLQHSVGTSPVW

SEQ ID NO: 27 - protein, 15 aa, Artificial Sequence
  Skin-Binding Peptide
QLSYHAYPQANHHAP

SEQ ID NO: 28 - protein, 14 aa, Artificial Sequence
  Skin-Binding Peptide
SGCHLVYDNGFCDH

SEQ ID NO: 29 - protein, 14 aa, Artificial Sequence
  Skin-Binding Peptide
ASCPSASHADPCAH

SEQ ID NO: 30 - protein, 14 aa, Artificial Sequence
  Skin-Binding Peptide
NLCDSARDSPRCKV

SEQ ID NO: 31 - protein, 12 aa, Artificial Sequence
  Skin-Binding Peptide
NHSNWKTAADFL

SEQ ID NO: 32 - protein, 12 aa, Artificial Sequence
  Skin-Binding Peptide
SDTISRLHVSMT

SEQ ID NO: 33 - protein, 12 aa, Artificial Sequence
  Skin-Binding Peptide
SPYPSWSTPAGR

SEQ ID NO: 34 - protein, 14 aa, Artificial Sequence
  Skin-Binding Peptide
DACSGNGHPNNCDR

SEQ ID NO: 35 - protein, 14 aa, Artificial Sequence
  Skin-Binding Peptide
DWCDTIIPGRTCHG

SEQ ID NO: 36 - protein, 8 aa, Artificial Sequence
  Caspase 3 cleavage site
LESGDEVD

SEQ ID NO: 37 - protein, 37 aa, Artificial Sequence
  Peptide Spacer
TSTSKASTTTTSSKTTTTSSKTTTTTSKTSTTSSSST

SEQ ID NO: 38 - protein, 22 aa, Artificial Sequence
  Peptide Spacer
GQGGYGGLGSQGAGRGGLGGQG

SEQ ID NO: 39 - protein, 10 aa, Artificial Sequence
  Peptide Spacer
GPGGYGPGQQ

SEQ ID NO: 40 - protein, 3 aa, Artificial Sequence
  Peptide spacer
GGG

SEQ ID NO: 41 - protein, 19 aa, Artificial Sequence
  Peptide having affinity for hair
PTPPELLHGAPRSKRKRKD

SEQ ID NO: 42 - protein, 19 aa, Artificial Sequence
  Peptide having affinity for hair
PEQISGSLVAAPWKRKRKD

SEQ ID NO: 43 - protein, 37 aa, Artificial Sequence
  Peptide having affinity for hair
PNTSQLSTGGGGHGHQKQHGLGHGHKHGHGHGHGHGK

SEQ ID NO: 44 - protein, 79 aa, Artificial Sequence
  Biologically expressed peptide
GSDPTPPELLHGAPRSKRKRKDPTPPELLHGAPRSKRKRKDPTPPELLHGAPRSKRKRKDPTPPELLHGAPRSKRKRKD

SEQ ID NO: 45 - DNA, 251 bp, Artificial Sequence
  Nucleotide sequence encoding peptide HC 71E
ggatccgacccaactccacctgaactgctgcatggtgctcctcgtagcaaacgtaagcgtaaagatccaactccgccggagctgctgcatggtgcaccgcgttccaaacgtaaacgcaaagacccgaccccaccagagctgctgcacggtgcgccgcgttctaaacgtaagcgcaaagatccgactccgccggaactgctgcacggcgcaccgcgctctaaacgtaaacgtaaagactaataaggcgcgcc

SEQ ID NO: 46 - protein, 79 aa, Artificial Sequence
  Biologically expressed peptide
GSDPEQISGSLVAAPWKRKRKDPEQISGSLVAAPWKRKRKDPEQISGSLVAAPWKRKRKDPEQISGSLVAAPWKRKRKD

SEQ ID NO: 47 - DNA, 251 bp, Artificial Sequence
  Nucleotide sequence encoding peptide HC 74E
ggatccgaccctgaacaaatttctggttctctggtagcagcaccttggaaacgcaaacgtaaagacccggaacaaatctctggcagcctggttgcagcgccgtggaagcgtaagcgcaaagatccggaacagattagcggttctctggtggccgctccgtggaagcgcaaacgtaaggacccggagcagatcagcggttctctggttgcggctccgtggaaacgtaagcgtaaagattaatgaggcgcgcc

SEQ ID NO: 48 - protein, 40 aa, Artificial Sequence
  Biologically expressed peptide
GSDPNTSQLSTGGGGHGHQKQHGLGHGHKHGHGHGHGHGK

SEQ ID NO: 49 - DNA, 134 bp, Artificial Sequence
  Nucleotide sequence encoding peptide HC 81E
ggatccgaccctaatacttctcaactgtctactggtggtggtggtcatggccaccagaaacagcatggtctgggccacggccacaaacacggccacggtcacggtcatggccacggcaaataatgaggcgcgcc

SEQ ID NO: 50 - DNA, 5388 bp, Artificial Sequence
  Plasmid pKSIC4-HC77623
agatctcgatcccgcgaaattaatacgactcactatagggagaccacaacggtttccctctagaaataattttgtttaactttaagaaggagatatacatatgcataccccagaacacatcaccgccgtggtacagcgctttgtggctgcgctcaatgccggcgatctggacggcatcgtcgcgctgtttgccgatgacgccacggtggaagagcccgtgggttccgagcccaggtccggtacggctgcgtgtcgtgagttttacgccaactcgctcaaactgcctttggcggtggagctgacgcaggagtgccgcgcggtcgccaacgaagcggccttcgctttcaccgtcagcttcgagtatcagggccgcaagaccgtagttgcgccctgtgatcactttcgcttcaatggcgccggcaaggtggtgagcatccgcgccttgtttggcgagaagaatattcacgcatgccagggatccgatccgactccgccgacgaatgtactgatgctggcaaccaaaggcggtggtacgcattccacgcacaaccatggcagcccgcgccacacgaatgctgacgcaggcaatccgggcggcggcaccccaccaaccaatgtcctgatgctggctactaaaggcggcggcacgcattctacccacaaccatggtagcccgcgccatactaatgcagatgccggcaacccgggcggtggtaccccgccaaccaacgttctgatgctggcgacgaaaggtggcggtacccattccacgcataatcatggcagccctcgccacaccaacgctgatgctggtaatcctggtggcggtaagaagaaataataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 51 - protein, 67 aa, Artificial Sequence
  Body surface-binding peptide block
PNTSQLSTGGGRTNAADHPKCGGGNTSQLSTGGGRTNAADHPKCGGGNTSQLSTGGGRTNAADHPKC

SEQ ID NO: 52 - protein, 55 aa, Artificial Sequence
  Body surface-binding peptide block
PRTNAADHPAAVTGGGCGGGRTNAADHPAAVTGGGCGGGRTNAADHPAAVTGGGC

SEQ ID NO: 53 - protein, 50 aa, Artificial Sequence
  Body surface-binding peptide block
PRTNAADHPAAVTGGGCGGGIPWWNIRAPLNAGGGCGGGDLTLPFHGGGC

SEQ ID NO: 54 - protein, 82 aa, Artificial Sequence
  Body surface-binding peptide block
PRTNAADHPGGGTPPELLHGDPRSKCGGGRTNAADHPGGGTPPELLHGDPRSKCGGGRTNAADHPGGGTPPELLHGDPRSKC

SEQ ID NO: 55 - protein, 82 aa, Artificial Sequence
  Body surface-binding peptide block
PTPPTNVLMLATKGGGRTNAADHPKCGGGTPPTNVLMLATKGGGRTNAADHPKCGGGTPPTNVLMLATKGGGRTNAADHPKC

SEQ ID NO: 56 - protein, 82 aa, Artificial Sequence
  Body surface-binding peptide block
PRTNAADHPGGGTPPTNVLMLATKKCGGGRTNAADHPGGGTPPTNVLMLATKKCGGGRTNAADHPGGGTPPTNVLMLATKKC

SEQ ID NO: 57 - protein, 12 aa, artificial sequence
  Peptide having affinity for hair
LDTSFHQVPFHQ

SEQ ID NO: 58 - protein, 18 aa, artificial sequence
  Peptide having affinity for hair
EQISGSLVAAPWEGEGER

SEQ ID NO: 59 - protein, 29 aa, artificial sequence
  Peptide having affinity for hair
PKRGRHKRPKRHKGGGTPPELLHGAPRSC

SEQ ID NO: 60 - protein, 18 aa, artificial sequence
  Peptide having affinity for hair
LDTSFHQVPFHQKRKRKD

SEQ ID NO: 61 - protein, 18 aa, artificial sequence
  Peptide having affinity for hair
EQISGSLVAAPWKRKRKD

SEQ ID NO: 62 - protein, 18 aa, artificial sequence
  Peptide having affinity for hair
TPPELLHGDPRSKRKRKD

SEQ ID NO: 63 - protein, 13 aa, artificial sequence
  Peptide having affinity for hair
NTSQLSTEGEGED

SEQ ID NO: 64 - protein, 20 aa, artificial sequence
  Peptide having affinity for PMMA
HTHHDTHKPWPTDAHRNSSV

SEQ ID NO: 65 - protein, 12 aa, artificial sequence
  Peptide having affinity for PMMA
IDTFYMSTMSHS

SEQ ID NO: 66 - protein, 15 aa, artificial sequence
  Peptide having affinity for hair
SSNYNYNYNYQYSSR

SEQ ID NO: 67 - protein, 21 aa, artificial sequence
  Peptide having affinity for hair
SSNYNYNYNYQYSSRKRKRKD

SEQ ID NO: 68 - protein, 21 aa, artificial sequence
  Peptide having affinity for hair
SSNYNYNYNYQYSSREGEGER

SEQ ID NO: 69 - protein, 76 aa, artificial sequence
  Peptide having affinity for hair
PGHTHHDTHKPWPTDAHRNSSVGAGHTHHDTHKPWPTDAHRNSSVGGSGPGSGGNTSQLSTGGGNTSQLSTGGPKK

SEQ ID NO: 70 - protein, 80 aa, artificial sequence
  Peptide having affinity for hair
PGIDTFYMSTMSHSGAGIDTFYMSTMSHSGGSGPGSGGNTSQLSTGSKGGGSKGGGSKGGGSKGGGSNTSQLSTGGPWKK

SEQ ID NO: 71 - DNA, 20 bp, artificial sequence
  Primer
ccctcatagttagcgtaacg

SEQ ID NO: 72 - protein, 20 aa, artificial sequence
  Tooth-binding peptide
AHPESLGIKYALDGNSDPHA

SEQ ID NO: 73 - protein, 20 aa, artificial sequence
  Tooth-binding peptide
ASVSNYPPIHHLATSNTTVN

SEQ ID NO: 74 - protein, 14 aa, artificial sequence
  Tooth-binding peptide
DECMEPLNAAHCWR

SEQ ID NO: 75 - protein, 14 aa, artificial sequence
  Tooth-binding peptide
DECMHGSDVEFCTS

SEQ ID NO: 76 - protein, 14 aa, artificial sequence
  Tooth-binding peptide
DLCSMQMMNTGCHY

SEQ ID NO: 77 - protein, 14 aa, artificial sequence
  Tooth-binding peptide
DLCSSPSTWGSCIR

SEQ ID NO: 78 - protein, 20 aa, artificial sequence
  Tooth-binding peptide
DPNESNYENATTVSQPTRHL

SEQ ID NO: 79 - protein, 20 aa, artificial sequence
  Tooth-binding peptide
EPTHPTMRAQMHQSLRSSSP

SEQ ID NO: 80 - protein, 20 aa, artificial sequence
  Tooth-binding peptide
GNTDTTPPNAVMEPTVQHKW

SEQ ID NO: 81 - protein, 15 aa, artificial sequence
  Tooth-binding peptide
NGPDMVQSVGKHKNS

SEQ ID NO: 82 - protein, 15 aa, artificial sequence
  Tooth-binding peptide
NGPEVRQIPANFEKL

SEQ ID NO: 83 - protein, 20 aa, artificial sequence
  Tooth-binding peptide
NNTSADNPPETDSKHHLSMS

SEQ ID NO: 84 - protein, 20 aa, artificial sequence
  Tooth-binding peptide
NNTWPEGAGHTMPSTNIRQA

SEQ ID NO: 85 - protein, 20 aa, artificial sequence
  Tooth-binding peptide
NPTATPHMKDPMHSNAHSSA

SEQ ID NO: 86 - protein, 20 aa, artificial sequence
  Tooth-binding peptide
NPTDHIPANSTNSRVSKGNT

SEQ ID NO: 87 - protein, 15 aa, artificial sequence
  Tooth-binding peptide
NPTDSTHMMHARNHE

SEQ ID NO: 88 - protein, 14 aa, artificial sequence
  Tooth-binding peptide
QHCITERLHPPCTK

SEQ ID NO: 89 - protein, 14 aa, artificial sequence
  Tooth-binding peptide
TPCAPASFNPHCSR

SEQ ID NO: 90 - protein, 14 aa, artificial sequence
  Tooth-binding peptide
TPCATYPHFSGCRA

SEQ ID NO: 91 - protein, 20 aa, artificial sequence
  Tooth-binding peptide
WCTDFCTRSTPTSTSRSTTS

SEQ ID NO: 92 - protein, 20 aa, artificial sequence
  Tooth-binding peptide
APPLKTYMQERELTMSQNKD

SEQ ID NO: 93 - protein, 20 aa, artificial sequence
  Tooth-binding peptide
EPPTRTRVNNHTVTVQAQQH

SEQ ID NO: 94 - protein, 14 aa, artificial sequence
  Tooth-binding peptide
GYCLRGDEPAVCSG

SEQ ID NO: 95 - protein, 20 aa, artificial sequence
  Tooth-binding peptide
LSSKDFGVTNTDQRTYDYTT

SEQ ID NO: 96 - protein, 14 aa, artificial sequence
  Tooth-binding peptide
NFCETQLDLSVCTV

SEQ ID NO: 97 - protein, 14 aa, artificial sequence
  Tooth-binding peptide
NTCQPTKNATPCSA

SEQ ID NO: 98 - protein, 20 aa, artificial sequence
  Tooth-binding peptide
PSEPERRDRNIAANAGRFNT

SEQ ID NO: 99 - protein, 18 aa, artificial sequence
  Tooth-binding peptide
THNMSHFPPSGHPKRTAT

SEQ ID NO: 100 - protein, 14 aa, artificial sequence
  Tooth-binding peptide
TTCPTMGTYHVCWL

SEQ ID NO: 101 - protein, 20 aa, artificial sequence
  Tooth-binding peptide
YCADHTPDPANPNKICGYSH

SEQ ID NO: 102 - protein, 20 aa, artificial sequence
  Tooth-binding peptide
AANPHTEWDRDAFQLAMPPK

SEQ ID NO: 103 - protein, 20 aa, artificial sequence
  Tooth-binding peptide
DLHPMDPSNKRPDNPSDLHT

SEQ ID NO: 104 - protein, 14 aa, artificial sequence
  Tooth-binding peptide
ESCVSNALMNQCIY

SEQ ID NO: 105 - protein, 20 aa, artificial sequence
  Tooth-binding peptide
HNKADSWDPDLPPHAGMSLG

SEQ ID NO: 106 - protein, 20 aa, artificial sequence
  Tooth-binding peptide
LNDQRKPGPPTMPTHSPAVG

SEQ ID NO: 107 - protein, 14 aa, artificial sequence
  Tooth-binding peptide
NTCATSPNSYTCSN

SEQ ID NO: 108 - protein, 14 aa, artificial sequence
  Tooth-binding peptide
SDCTAGLVPPLCAT

SEQ ID NO: 109 - protein, 20 aa, artificial sequence
  Tooth-binding peptide
TIESSQHSRTHQQNYGSTKT

SEQ ID NO: 110 - protein, 20 aa, artificial sequence
  Tooth-binding peptide
VGTMKQHPTTTQPPRVSATN

SEQ ID NO: 111 - protein, 20 aa, artificial sequence
  Tooth-binding peptide
YSETPNDQKPNPHYKVSGTK

(19) United States
(12) Patent Application Publication
(10) Pub. No.: US 2008/0280810 A1
(43) Pub. Date: Nov. 13, 2008

(54) PEPTIDES HAVING AFFINITY FOR BODY SURFACES

(76) Inventors: JOHN P. O'BRIEN, Oxford, PA (US); Hong Wang, Kennett Square, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/877,692

(22) Filed: Oct. 24, 2007

Related U.S. Application Data
(60) Provisional application No. 60/855,251, filed on Oct. 30, 2006.

(51) Int. Cl. A61K 38/02; C07K 2/00; A61Q 5/00

(52) U.S. Cl. 514/2; 530/300

(57) ABSTRACT

Peptides having affinity for a body surface are provided. The peptides comprise a body surface-binding peptide block and at least one charged, terminal peptide block. These peptides have enhanced affinity for body surfaces and deposit more rapidly onto body surfaces than peptides lacking the charged terminal groups. The peptides are used to deliver and/or to seal benefit agents to body surfaces, thereby providing enhanced durability.

This application claims the benefit of U.S. Provisional Application No. 60/855,251 filed Oct. 30, 2006.

### FIELD OF THE INVENTION

The invention pertains to the field of personal care. More specifically, the invention provides peptides having affinity for a body surface comprising a body surface-binding peptide block and at least one charged, terminal peptide block. The invention also provides peptide-based body surface reagents comprising a peptide having affinity for a body surface coupled to a benefit agent.

### BACKGROUND OF THE INVENTION

Benefit agents for hair, skin, and oral cavity surfaces are well-known and frequently used components of personal care products. One major problem with many of these benefit agents is that they lack the required durability for long-lasting effects. Oxidative hair dyes provide long-lasting color, but the oxidizing agents they contain cause hair damage. In order to improve the durability of hair and skin care compositions, peptide-based hair and skin conditioners, colorants, and other benefit agents have been developed (Huang et al., commonly owned U.S. Pat. No. 7,220,405, and U.S. Patent Application Publication No. 2005/0226839). Peptide-based sunscreens have also been described (Buseman-Williams et al., co-pending and commonly owned U.S. Patent Application Publication No. 2005/0249682; and Lowe et al., co-pending and commonly owned U.S. Patent Application Publication No. 2007/0110686).

The peptide-based benefit agents are prepared by coupling a specific peptide sequence that has a high binding affinity to a body surface, such as hair, skin, or tissues of the oral cavity (e.g., gums, teeth (enamel or pellicle), tongue, etc.), with a benefit agent. The peptide portion binds to the body surface, thereby strongly attaching the benefit agent. Additionally, the use of hair and skin-binding peptides as sealants to enhance the durability of benefit agents has been described (Beck et. al. co-pending and commonly owned U.S. Patent Application Publication No. 2007/0067924).

Peptides with a high binding affinity to body surfaces, such as hair skin, fingernails, and oral cavity surfaces, have been identified using phage display screening techniques (Huang et al., U.S. Pat. No. 7,220,405, and U.S. Patent Application Publication No. 2005/0226839; Estell et al. WO 01/79479; Murray et al., U.S. Patent Application Publication No. 2002/0098524; Janssen et al., U.S. Patent Application Publication No. 2003/0152976; and Janssen et al., WO 2004/048399). Additionally, empirically-generated hair and skin-binding peptides that are based on charged amino acids have been reported (Rothe et al., WO 2004/000257). These body surface-binding peptides provide a means to deliver benefit agents to a body surface, resulting in improved durability; however, body surface-binding peptides with stronger affinity for body surfaces are needed for even greater durability under harsh conditions (e.g., shampooing, and washing). Additionally, peptides that bind to a body surface at a faster rate are needed to reduce application times.

O'Brien, in co-pending and commonly owned U.S. Patent Application Publication No. 2007/0022547, describes affinity peptides comprising at least one positively charged amino acid residue at the N-terminal and/or C-terminal end of the sequence of a peptide having binding affinity for pigment or substrate surfaces. The addition of the positively charged amino acids to the binding sequence significantly enhances the strength of the interaction of the peptide with substrate surfaces such as paper and textile fabrics. However, peptides comprising a body surface-binding peptide block and at least one charged, terminal peptide block were not described in that disclosure.

The problem to be solved, therefore, is to provide body surface-binding peptides that have a higher affinity to body surfaces and that deposit onto body surfaces at a faster rate than peptides known in the art.

Applicants have addressed the stated problem by discovering that the addition of a charged peptide block to the N-terminal and/or C-terminal end of a body surface-binding peptide sequence enhances the affinity of the peptide for the body surface and increases the rate at which the peptide is deposited onto the body surface.

### SUMMARY OF THE INVENTION

The invention provides peptides having enhanced affinity for a body surface which comprise a body surface-binding peptide block and at least one terminal charged peptide block. The peptides having affinity for a body surface can be coupled to various benefit agents to provide peptide-based body surface reagents, which are used to deliver a benefit agent to a body surface.
Accordingly the invention provides a peptide having affinity for a body surface having the general structure:

(nCPB^{±})ₓ-(Sn)-BSBPB-(Sc)-(cCPB^{±})_{y}
wherein:
   (i) nCPB^{±} is an N-terminal charged peptide block, said N-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
   (ii) cCPB^{±} is a C-terminal charged peptide block, said C-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
   (iii) BSBPB is a body surface-binding peptide block comprising at least one body surface-binding peptide;
   (iv) x and y are independently 0 or 1, provided that x and x may not both be 0; and
   (v) Sn and Sc are optional peptide spacers comprised of 0 to about 20 amino acids.

In another embodiment the invention provides a peptide-based body surface reagent comprising a peptide having affinity for a body surface coupled to benefit agent, said peptide-based body surface reagent having the general structure:

{(nCPB^{±})ₓ-(Sn)-BSBPB-(Sc)-(cCPB^{±})_{y}}_{z}-(So-BAₛ)ᵣ,

wherein:
(i) nCPB^{±} is an N-terminal charged peptide block, said N-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(ii) cCPB^{±} is a C-terminal charged peptide block, said C-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(iii) BSBPB is a body surface-binding peptide block comprising at least one body surface-binding peptide;
(iv) BA is a benefit agent;
(v) x and y are independently 0 or 1, provided that x and x may not both be 0;
(vi) z=1 to about 10,000;
(vii) r and s are independently 1 to about 100;
(viii) So is an optional organic spacer; and
(ix) Sn and Sc are optional peptide spacers comprised of 0 to about 20 amino acids.

In preferred embodiments of the invention the body surface binding peptide has affinity for a body surface has affinity for a body surface selected from the group consisting of hair, nails, teeth, gums, skin, and tissues of the oral cavity.

Alternatively the peptide having affinity for a body surface comprises at least one body surface-binding peptide which is isolated by a process comprising the steps of:
(i) providing a library of combinatorially generated phage-peptides;
(ii) contacting the library of (i) with a body surface to form a reaction solution comprising:
   (A) phage-peptide-body surface complex;
   (B) unbound body surface, and
   (C) uncomplexed peptides;
(iii) isolating the phage-peptide-body surface complex of (ii);
(iv) eluting the weakly bound peptides from the isolated peptide complex of (iii); and
(v) identifying the remaining bound phage-peptides either by using the polymerase chain reaction directly with the phage-peptide-body surface complex remaining after step (iv), or by infecting bacterial host cells directly with the phage-peptide-body surface complex remaining after step (iv), growing the infected cells in a suitable growth medium, and isolating and identifying the phage-peptides from the grown cells.

In another embodiment the invention provides a method for applying a benefit agent to a body surface comprising the steps of:
a) providing a composition comprising a peptide-based body surface reagent according to the invention; and
b) applying the composition to the body surface for a time sufficient for the peptide-based body surface reagent to bind to the body surface.

Alternatively the invention provides a method for applying a benefit agent to a body surface comprising the steps of:
a) providing a benefit agent having affinity to the body surface;
b) providing a composition comprising a peptide having affinity for a body surface of the invention and
c) applying the benefit agent and said composition to the body surface for a time sufficient for the benefit agent and the peptide having affinity for the body surface or the peptide based body surface reagent to bind to the body surface.

In a specific embodiment, the invention provides peptides having affinity for a body surface selected from the group consisting of SEQ ID NOs: 41, 42, 43, 58, 59, 60, 61, 62, 63, 69, and 70.

In another embodiment, combinatorially generated tooth-binding peptides are provided selected from the group consisting of SEQ ID NOs: 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, and 111.

### BRIEF DESCRIPTION OF FIGURES AND SEQUENCE DESCRIPTIONS

The various embodiments of the invention can be more fully understood from the following detailed description, figure, and the accompanying sequence descriptions, which form a part of this application.

FIG. 1 is a plasmid map of the vector pKSIC4-HC77623, described in Examples 1-3.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-7, and 15-23 are the amino acid sequences of hair-binding peptides.

SEQ ID NOs:8-12, and 24-35 are the amino acid sequences of skin-binding peptides.

SEQ ID NOs:13 and 14 are the amino acid sequences of nail-binding peptides.

SEQ ID NO:36 is the amino acid sequence of the Caspase 3 cleavage site.

SEQ ID NOs:37-40 are the amino acid sequences of peptide spacers.

SEQ ID NO:41-43 are the amino acid sequences of peptides having affinity for hair which comprise a hair-binding peptide block and a charged terminal peptide block.

SEQ ID NOs:44, 46, and 48 are the amino acid sequences of the biologically expressed peptides described in Examples 1-3.

SEQ ID NOs:45, 47, and 49 are the nucleotide sequences of genes that encode the biologically expressed peptides described in Examples 1-3.

SEQ ID NO: 50 is the nucleotide sequence of plasmid pKSIC4—HC77623, described in Examples 1-3.

SEQ ID NOs:51-56 are the amino acid sequences of body surface-binding peptide blocks comprising multiple hair-binding peptides.

SEQ ID NO:57 is the amino acid sequence of a hair-binding peptide referred to as “F4”.

SEQ ID NOs:58-63 are the amino acid sequences of peptides having affinity for hair which comprise a hair-binding peptide block and a charged terminal peptide block.

SEQ ID NOs:64-65 are the amino acid sequences of polymethylmethacrylate (PMMA) binding peptides.

SEQ ID NO:66 is the amino acid sequence of a dyed-hair-binding peptide.

SEQ ID NO:67-68 are the amino acid sequences of peptides having affinity for dyed hair which comprise a dyed-hair-binding peptide block and a charged terminal peptide block.

SEQ ID NOs: 69-70 are the amino acid sequence of multi-block peptides having affinity for hair which comprise at least one hair-binding block, at least one PMMA-binding block, and a charged terminal peptide block.

SEQ ID NO: 71 is the nucleic acid sequence of sequencing primer.

SEQ ID NOs:72-111 are the amino acid sequences of tooth-binding peptides. SEQ ID NOs: 72-91 bind to tooth pellicle. SEQ ID NOs: 92-111 bind to tooth enamel.

### DETAILED DESCRIPTION OF THE INVENTION

The present invention provides peptides having affinity for a body surface comprising a body surface-binding peptide block and at least one charged, terminal peptide block. These peptides bind with higher affinity to body surfaces and deposit onto body surfaces at a faster rate than body surface-binding peptides known in the art. The peptides having affinity for a body surface can be coupled to various benefit agents to provide peptide-based body surface reagents, which are used to deliver the benefit agent to the body surface. Alternatively, the peptides disclosed herein may be used as a sealant to enhance the durability of benefit agents.

The following definitions are used herein and should be referred to for interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

“BSBP” means body surface-binding peptide.

“BSBPB” means body surface-binding peptide block.

“HBP” means hair-binding peptide.

“SBP” means skin-binding peptide.

“NBP” means nail-binding peptide.

“OBP” means oral cavity surface-binding peptide.

“TBP” means a tooth-binding peptide.

“BA” means benefit agent.

“HCA” means hair conditioning agent.

“SCA” means skin conditioning agent.

“OCBA” means oral cavity benefit agent.

“C” means colorant.

The terms “nCPB^{±}” and “N-terminal charged peptide block” refer to a charged peptide block at the N-terminal end of a body surface-binding peptide. The N-terminal charged peptide block comprises at least 30 mole %, and alternatively at least 50 mole %, and alternatively at least 75 mole %, and further alternatively 100 mole % of charged amino acids selected from lysine, arginine, histidine, aspartic acid, glutamic acid, and combinations thereof. The N-terminal charged peptide block is from 1 to about 50 amino acids in length.

The terms “cCPB^{±}” and “C-terminal charged peptide block” refer to a charged peptide block at the C-terminal end of a body surface-binding peptide. The C-terminal charged peptide block comprises at least 30 mole %, and alternatively at least 50 mole %, and alternatively at least 75 mole %, and further alternatively 100 mole % of charged amino acids selected from lysine, arginine, histidine, aspartic acid, glutamic acid, and combinations thereof. The C-terminal charged peptide block is from 1 to about 50 amino acids in length.

“Sn” refers to an optional peptide spacer at the N-terminus of the body surface-binding peptide block of the peptides disclosed herein.

“Sc” refers to an optional peptide spacer at the C-terminus of the body surface-binding peptide block of the peptides disclosed herein.

“So” refers to an optional organic spacer that may be used to link the peptide having affinity for a body surface with a benefit agent.

“S” refers to an optional peptide spacer that links two body surface-binding peptides together in the body surface-binding peptide block.

The term “peptide” refers to two or more amino acids joined to each other by peptide bonds or modified peptide bonds.

The term “body surface-binding peptide” refers to peptides that bind with high affinity to a body surface. The body surface-binding peptides of the invention are from about 7 amino acids to about 50 amino acids, more preferably, from about 7 amino acids to about 25 amino acids, most preferably from about 7 to about 20 amino acids in length.

The term “body surface-binding peptide block” refers to a peptide that comprises at least one body surface-binding peptide. Where the body surface-binding peptide block comprises two or more body surface-binding peptides, the body surface-binding peptide peptides may be linked together either directly or through a peptide spacer (S).

The term “hair-binding peptide” refers to peptides that bind with high affinity to hair. The hair-binding peptides of the invention are from about 7 amino acids to about 50 amino acids, more preferably, from about 7 amino acids to about 25 amino acids, most preferably from about 7 to about 20 amino acids in length.

The term “skin-binding peptide” refers to peptides that bind with high affinity to skin. The skin-binding peptides of the invention are from about 7 amino acids to about 50 amino acids, more preferably, from about 7 amino acids to about 25 amino acids, most preferably from about 7 to about 20 amino acids in length.

The term “nail-binding peptide” refers to peptides that bind with high affinity to fingernails or toenails. The nail-binding peptides of the invention are from about 7 amino acids to about 50 amino acids, more preferably, from about 7 amino acids to about 25 amino acids, most preferably from about 7 to about 20 amino acids in length.

The term “oral cavity surface-binding peptide” refers to peptides that bind with high affinity to teeth, gums, cheeks, tongue, or other surfaces in the oral cavity. In one embodiment, the oral cavity surface-binding peptide is a peptide that binds with high affinity to teeth. In a further embodiment, the oral cavity surface-binding peptide is a peptide that binds with high affinity to tooth enamel and/or the tooth pellicle. The oral cavity-binding peptides of the invention are from about 7 amino acids to about 50 amino acids, more preferably, from about 7 amino acids to about 25 amino acids, most preferably from about 7 to about 20 amino acids in length.

The term “benefit agent” is a general term that refers to an agent that provides a cosmetic or prophylactic effect when applied to a body surface, such as skin, hair or surfaces of the oral cavity. Benefit agents typically include conditioning agents, colorants, fragrances, whiteners, sunscreen agents, and oral benefit agents, such as white colorants, whitening agents, enzymes, anti-plaque agents, anti-staining agents, anti-microbial agents, anti-caries agents, flavoring agents, coolants, and salivating agents; along with other substances commonly used in the personal care industry.

The term “body surface” means any surface of the human body that may serve as a substrate for the binding of a peptide. Typical body surfaces include, but are not limited to, hair, skin, nails, teeth, gums, and various tissues of the oral cavity. In one embodiment, skin and/or hair-binding peptides may also bind to keratinaceous surfaces of the oral cavity.

The term “hair” as used herein refers to human hair, eyebrows, and eyelashes.

The term “skin” as used herein refers to human skin, or substitutes for human skin, such as pig skin, VITRO-SKIN® and EPIDERM™. Skin as used herein as a body surface will generally comprise a layer of epithelial cells and may additionally comprise a layer of endothelial cells.

The term “nails” as used herein refers to human fingernails and toenails.

The term “tooth surface” will refer to both tooth enamel and tooth pellicle surfaces of mammalian teeth. In a preferred embodiment, the tooth surface will refer to both tooth enamel and tooth pellicle surfaces of human teeth. As such, both tooth enamel-binding peptides and tooth pellicle-binding peptides will be collectively referred to as tooth-binding peptides.

The terms “coupling” and “coupled” as used herein refer to any chemical association and include both covalent and non-covalent interactions.

The term “stringency” as it is applied to the selection of body surface-binding peptides of the present invention, refers to the concentration of the eluting agent (usually detergent) used to elute peptides from a body surface. Higher concentrations of the eluting agent provide more stringent conditions.

The term “peptide-body surface complex” means a structure comprising a peptide bound to a sample of a body surface via a binding site on the peptide.

The term “peptide-hair complex” means a structure comprising a peptide bound to a hair fiber via a binding site on the peptide.

The term “peptide-skin complex” means a structure comprising a peptide bound to the skin via a binding site on the peptide.

The term “peptide-nail complex” means a structure comprising a peptide bound to a fingernail or toenail via a binding site on the peptide.

The term “peptide-tooth complex” means a structure comprising a peptide bound to a tooth via a binding site on the peptide. In one embodiment, the peptide-tooth complex means a structure comprising a peptide bound to a tooth enamel surface or a peptide bound to a tooth pellicle surface.

The term “phage-peptide-body surface complex” means a structure comprising a phage displayed peptide bound to a body surface via a binding site on the displayed peptide.

As used herein, the terms “pellicle” and “tooth pellicle” will refer to the thin film (typically 1 to 10 μm thick) derived from salivary glycoproteins which forms over the surface of the tooth crown.

As used herein, the terms “enamel” and “tooth enamel” will refer to the highly mineralized tissue which forms the outer layer of the tooth. The enamel layer is composed primarily of crystalline calcium phosphate (i.e. hydroxyapatite) along with water and some organic material.

The term “nanoparticles” are herein defined as particles with an average particle diameter of between 1 and 500 nm. Preferably, the average particle diameter of the particles is between about 1 and 200 nm. As used herein, “particle size” and “particle diameter” have the same meaning. Nanoparticles include, but are not limited to, metallic, semiconductor, polymer, or other organic or inorganic particles.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any (or as defined herein) | Xaa | X |

As used herein, the term “gene” refers to a nucleic acid fragment that expresses a specific protein, optionally including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

In one aspect, the invention provides peptides having affinity for a body surface comprising a body surface-binding peptide block and at least one charged terminal peptide block. Body surface-binding peptides have affinity for a body surface, such as hair, skin, nails, or oral cavity surfaces and may be identified using combinatorial methods, such as phage display. The peptides of the invention are formed by linking at least one charged terminal peptide block to the body surface-binding peptide sequence. The peptides of the invention can be coupled to benefit agents to produce peptide-based body surface reagents, which are used to deliver the benefit agents to the body surface. Additionally, the peptides and the peptide-based body surface reagents can be used as sealants to enhance the durability of benefit agents on the body surface.

### Body Surfaces

Body surfaces are any surface on the human body that will serve as a substrate for a binding peptide. Typical body surfaces include, but are not limited to, hair, skin, nails, teeth, gums, and the tissues of the oral cavity. In many cases the body surfaces of the invention will be exposed to air, however in some instances, the oral cavity for example, the surfaces will be internal. Accordingly, body surfaces may include layers of both epithelial and endothelial cells.

Samples of body surfaces are available from a variety of sources. For example, human hair samples are available commercially from companies such as International Hair Importers and Products (Bellerose, N.Y.), in different colors, such as brown, black, red, and blond, and in various types, such as African-American, Caucasian, and Asian. Additionally, the hair samples may be treated, for example, using hydrogen peroxide to obtain bleached hair. Human skin samples may be obtained from cadavers or in vitro human skin cultures. Alternatively, pig skin, available from butcher shops and supermarkets, VITRO-SKIN®, available from IMS Inc. (Milford, Conn.), and EPIDERM™, available from MatTek Corp. (Ashland, Mass.), may be used as substitutes for human skin. Human fingernails and toenails may be obtained from volunteers. Extracted human teeth may be obtained from dental offices. In one embodiment, the tooth surface includes, but it not limited to tooth enamel and tooth pellicle. Additionally, hydroxyapatite, available in many forms, for example, from Berkeley Advanced Biomaterials, Inc. (San Leandro, Calif.), may be used (once coated with salivary glycoproteins to form an acquired pellicle) as a model for studying teeth-binding peptides.

### Body Surface-Binding Peptides

Body surface-binding peptides as defined herein are peptide sequences that bind with high affinity to specific body surfaces, including, but not limited to, hair, skin, nails, teeth, tongue, cheeks, lips, gums, and the tissues of the oral cavity, for example. Body surface-binding peptides of the present invention are from about 7 amino acids to about 50 amino acids, more preferably, from about 7 amino acids to about 25 amino acids, and most preferably from about 7 to about 20 amino acids in length.

Suitable body surface-binding peptide sequences may be selected using methods that are well known in the art. Specifically, the body surface-binding peptides can be generated randomly and then selected against a specific body surface, for example, hair, skin, nail, or oral cavity surface sample, based upon their binding affinity for the surface of interest. The generation of random libraries of peptides is well known and may be accomplished by a variety of techniques including, bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display technology (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Exemplary methods used to generate such biological peptide libraries are described in Dani, M., J. of Receptor & Signal Transduction Res., 21 (4):447-468 (2001), Sidhu et al., Methods in Enzymology 328:333-363 (2000), and Phage Display of Peptides and Proteins, A Laboratory Manual, Brian K. Kay, Jill Winter, and John McCafferty, eds.; Academic Press, NY, 1996. Additionally, phage display libraries are available commercially from companies such as New England Biolabs (Beverly, Mass.).

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

More specifically, after a suitable library of peptides has been generated or purchased, the library is then contacted with an appropriate amount of the test substrate, specifically a body surface sample. The library of peptides is dissolved in a suitable solution for contacting the sample. The body surface sample may be suspended in the solution or may be immobilized on a plate or bead. A preferred solution is a buffered aqueous saline solution containing a surfactant. A suitable solution, for example, is Tris-buffered saline (TBS) with 0.5% TWEEN® 20. The solution may additionally be agitated by any means in order to increase the mass transfer rate of the peptides to body surface sample, thereby shortening the time required to attain maximum binding.

Upon contact, a number of the randomly generated peptides will bind to the body surface sample to form a peptide-body-surface complex, for example a peptide-hair, peptide-skin, peptide-nail, or peptide-tooth complex. Unbound peptide may be removed by washing. After all unbound material is removed, peptides having varying degrees of binding affinities for the test surface may be fractionated by selected washings in buffers having varying stringencies. Increasing the stringency of the buffer increases the required strength of the bond between the peptide and body surface in the peptide-body surface complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that peptides having increasing binding affinities for body surface substrates may be eluted by repeating the selection process using buffers with increasing stringencies. The eluted peptides can be identified and sequenced by any means known in the art.

Thus, the following method for generating the body surface-binding peptides, for example, hair-binding peptides, skin-binding peptides, nail-binding peptides, or tooth-binding peptides, may be used. A library of combinatorially generated phage-peptides is contacted with the body surface of interest, to form phage-peptide-body surface complexes. The phage-peptide-body surface complex is separated from the uncomplexed phage-peptides and unbound substrate, and the bound phage-peptides from the phage-peptide-body surface complexes are eluted from the complexes, preferably by acid treatment. Then, the eluted phage-peptides are identified and sequenced. To identify peptide sequences that bind to one body surface but not to another (non-target body surface), for example, peptides that bind to hair, but not to skin or peptides that bind to skin, but not to hair, a subtractive panning step is added. Specifically, the library of combinatorially generated phage-peptides is first contacted with the non-target body surface to remove phage-peptides that bind to it. Then, the unbound phage-peptides are contacted with the desired body surface and the above process is followed. Alternatively, the library of combinatorially generated phage-peptides may be contacted with the non-target body surface and the desired body surface simultaneously. Then, the phage-peptide-body surface complexes are separated from the phage-peptide-non-target body surface complexes and the method described above is followed for the desired phage-peptide-body surface complexes.

In one embodiment, a modified phage display screening method for isolating peptides with a higher affinity for body surfaces is used. In the modified method, the phage-peptide-body surface complexes are formed as described above. Then, these complexes are treated with an elution buffer. Any of the elution buffers described above may be used. Preferably, the elution buffer is an acidic solution. Then, the remaining, elution-resistant phage-peptide-body surface complexes are used to directly infect a bacterial host cell, such as E. coli ER2738. The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-Gal™. After growth, the plaques are picked for DNA isolation and are sequenced to identify the peptide sequences with a high binding affinity for the body surface of interest.

In another embodiment, PCR may be used to identify the elution-resistant phage-peptides from the modified phage display screening method as described above, by directly carrying out PCR on the phage-peptide-body surface complexes using the appropriate primers, as described by Janssen et al. in U.S. Patent Application Publication No. 2003/0152976.

Body surface-binding peptide sequences have been identified using the above methods, as described by Huang et al. in co-pending and commonly owned U.S. Pat. No. 7,220,405, and U.S. Patent Application Publication No. 2005/0226839. Examples of suitable combinatorially generated body surface-binding peptides include, but are not limited to, hair-binding sequences, given as SEQ ID NOs:1-7, 15-23, and 57; skin-binding sequences, given as SEQ ID NOs:8-12 and 24-35; fingernail-binding peptide sequences SEQ ID NOs:13 and 14; and tooth binding peptides SEQ ID NOs: 72-111 (See Table A).

In a further embodiment, combinatorially generated tooth binding peptides are provided selected from the group consisting of SEQ ID NOs: 72-111.

Moreover, shampoo-resistant hair-binding peptides may be selected using a modified biopanning method as described by O'Brien et al. in co-pending and commonly owned U.S. Patent Application Publication No. 2006/0073111. Similarly, hair conditioner-resistant hair-binding peptides and skin care composition resistant skin-binding peptides may be identified using the methods described by Wang et al. (co-pending and commonly owned U.S. Patent Application Publication No. 2007/0196305) and Wang et al. (co-pending and commonly owned U.S. Patent Application Publication No. 2006/0199206), respectively. In those methods, either the initial library of phage peptides is dissolved in the matrix of interest (i.e., a shampoo matrix, a hair conditioner matrix or a skin care composition matrix) for contacting with the substrate, or the peptide-body surface complex, after it is formed by contacting the body surface with the library of phage peptides, as described above, is contacted with the matrix of interest. The biopanning method is then conducted as described above. The shampoo matrix, hair conditioner matrix, or skin care composition matrix may be a full strength commercial product or a dilution thereof. Suitable examples of shampoo resistant hair-binding peptides and/or conditioner-resistant hair-binding peptides, and skin care composition-resistant skin-binding peptides are given as SEQ ID NOs:15-23, and SEQ ID NOs:24-35, respectively (see Table A).

Similarly, the matrix of interest may be an oral care composition matrix [toothpaste, mouthwash, gum, polishing compounds, etc. and orally acceptable components thereof]. As such, the initial library of phage peptides may be dissolved in the oral care matrix for contacting the substrate (oral cavity surface), or the peptide-oral cavity surface complex, after it is formed by contacting the oral cavity surface with the library of phage peptides is contacted with the matrix of interest.

Additionally, any body surface-binding peptide known in the art may be used, such as those reported by Estell et al. (WO 0179479); Murray et al., (U.S. Patent Application Publication No. 2002/0098524); and Janssen et al., (U.S. Patent Application Publication No. 2003/0152976).

Body surface-binding peptide sequences may also be determined using the method described by Lowe in co-pending and commonly owned U.S. Patent Application Publication No. 2006/0286047. That method provides a means for determining the sequence of a peptide binding motif having affinity for a particular substrate, for example a body surface. First, a population of binding peptides for the substrate of interest is identified by biopanning using a combinatorial method, such as phage display. Rather than using many rounds of biopanning to identify specific binding peptide sequences and then using standard pattern recognition techniques to identify binding motifs, as is conventionally done in the art, the method requires only a few rounds of biopanning. The sequences in the population of binding peptides, which are generated by biopanning, are analyzed by identifying subsequences of 2, 3, 4, and 5 amino acid residues that occur more frequently than expected by random chance. The identified subsequences are then matched head to tail to give peptide motifs with substrate binding properties. This procedure may be repeated many times to generate long peptide sequences.

TABLE A

Examples of Body Surface-Binding Peptide
Sequences

| | | |
|---|---|---|
| | SEQ | |
| | ID | |
| Body Surface | NO: | Sequence |

| | | |
|---|---|---|
| Hair | 1 | RTNAADHPAAVT |
| Hair | 2 | DLTLPFH |
| Hair | 3 | THSTHNHGSPRHTNADAGNP |
| Hair | 4 | LPRIANTWSPS |
| Hair | 5 | EQISGSLVAAPW |
| Hair | 6 | TDMQAPTKSYSN |
| Hair | 7 | LDTSFPPVPFHA |
| Hair (Shampoo | 15 | TPPTNVLMLATK |
| Resistant) | | |
| Hair (Shampoo | 16 | TPPELLHGDPRS |
| Resistant) | | |
| Hair (Shampoo | 17 | TPPELLHGAPRS |
| Resistant) | | |
| Hair (Shampoo | 18 | NTSQLST |
| Resistant) | | |
| Hair | 19 | STLHKYKSQDPTPHH |
| (Conditioner | | |
| Resistant) | | |
| Hair (Shampoo | 20 | GMPAMHWIHPFA |
| and Conditioner | | |
| Resistant) | | |
| Hair (Shampoo | 21 | HDHKNQKETHQRHAA |
| and Conditioner | | |
| Resistant) | | |
| Hair (Shampoo | 22 | HNHMQERYTDPQHSPSVNGL |
| and Conditioner | | |
| Resistant) | | |
| Hair (Shampoo | 23 | TAEIQSSKNPNPHPQRSWTN |
| and Conditioner | | |
| Resistant) | | |
| Skin | 8 | TPFHSPENAPGS |
| Skin | 9 | FTQSLPR |
| Skin | 10 | KQATFPPNPTAY |
| Skin | 11 | HGHMVSTSQLSI |
| Skin | 12 | LSPSRMK |
| Skin | 24 | SVSVGMKPSPRP |
| (Body Wash | | |
| Resistant) | | |
| Skin | 25 | TMGFTAPRFPHY |
| (Body Wash | | |
| Resistant) | | |
| Skin | 26 | NLQHSVGTSPVW |
| (Body Wash | | |
| Resistant) | | |
| Skin | 27 | QLSYHAYPQANHHAP |
| (Body Wash | | |
| Resistant) | | |
| Skin | 28 | SGCHLVYDNGFCDH |
| (Body Wash | | |
| Resistant) | | |
| Skin | 29 | ASCPSASHADPCAH |
| (Body Wash | | |
| Resistant) | | |
| Skin | 30 | NLCDSARDSPRCKV |
| (Body Wash | | |
| Resistant) | | |
| Skin | 31 | NHSNWKTAADFL |
| (Body Wash | | |
| Resistant) | | |
| Skin | 32 | SDTISRLHVSMT |
| (Body Wash | | |
| Resistant) | | |
| Skin | 33 | SPYPSWSTPAGR |
| (Body Wash | | |
| Resistant) | | |
| Skin | 34 | DACSGNGHPNNCDR |
| (Body Wash | | |
| Resistant) | | |
| Skin | 35 | DWCDTIIPGRTCHG |
| (Body Wash | | |
| Resistant) | | |
| Fingernail | 13 | ALPRIANTWSPS |
| Fingernail | 14 | YPSFSPTYRPAF |
| Hair | 57 | LDTSFHQVPFHQ |
| Tooth (pellicle) | 72 | AHPESLGIKYALDGNSDPHA |
| Tooth (pellicle) | 73 | ASVSNYPPIHHLATSNTTVN |
| Tooth (pellicle) | 74 | DECMEPLNAAHCWR |
| Tooth (pellicle) | 75 | DECMHGSDVEFCTS |
| Tooth (pellicle) | 76 | DLCSMQMMNTGCHY |
| Tooth (pellicle) | 77 | DLCSSPSTWGSCIR |
| Tooth (pellicle) | 78 | DPNESNYENATTVSQPTRHL |
| Tooth (pellicle) | 79 | EPTHPTMRAQMHQSLRSSSP |
| Tooth (pellicle) | 80 | GNTDTTPPNAVMEPTVQHKW |
| Tooth (pellicle) | 81 | NGPDMVQSVGKHKNS |
| Tooth (pellicle) | 82 | NGPEVRQIPANFEKL |
| Tooth (pellicle) | 83 | NNTSADNPPETDSKHHLSMS |
| Tooth (pellicle) | 84 | NNTWPEGAGHTMPSTNIRQA |
| Tooth (pellicle) | 85 | NPTATPHMKDPMHSNAHSSA |
| Tooth (pellicle) | 86 | NPTDHIPANSTNSRVSKGNT |
| Tooth (pellicle) | 87 | NPTDSTHMMHARNHE |
| Tooth (pellicle) | 88 | QHCITERLHPPCTK |
| Tooth (pellicle) | 89 | TPCAPASFNPHCSR |
| Tooth (pellicle) | 90 | TPCATYPHFSGCRA |
| Tooth (pellicle) | 91 | WCTDFCTRSTPTSTSRSTTS |
| Tooth (enamel) | 92 | APPLKTYMQERELTMSQNKD |
| Tooth (enamel) | 93 | EPPTRTRVNNHTVTVQAQQH |
| Tooth (enamel) | 94 | GYCLRGDEPAVCSG |
| Tooth (enamel) | 95 | LSSKDFGVTNTDQRTYDYTT |
| Tooth (enamel) | 96 | NFCETQLDLSVCTV |
| Tooth (enamel) | 97 | NTCQPTKNATPCSA |
| Tooth (enamel) | 98 | PSEPERRDRNIAANAGRFNT |
| Tooth (enamel) | 99 | THNMSHFPPSGHPKRTAT |
| Tooth (enamel) | 100 | TTCPTMGTYHVCWL |
| Tooth (enamel) | 101 | YCADHTPDPANPNKICGYSH |
| Tooth (enamel) | 102 | AANPHTEWDRDAFQLAMPPK |
| Tooth (enamel) | 103 | DLHPMDPSNKRPDNPSDLHT |
| Tooth (enamel) | 104 | ESCVSNALMNQCIY |
| Tooth (enamel) | 105 | HNKADSWDPDLPPHAGMSLG |
| Tooth (enamel) | 106 | LNDQRKPGPPTMPTHSPAVG |
| Tooth (enamel) | 107 | NTCATSPNSYTCSN |
| Tooth (enamel) | 108 | SDCTAGLVPPLCAT |
| Tooth (enamel) | 109 | TIESSQHSRTHQQNYGSTKT |
| Tooth (enamel) | 110 | VGTMKQHPTTTQPPRVSATN |
| Tooth (enamel) | 111 | YSETPNDQKPNPHYKVSGTK |

### Peptides Having Affinity for a Body Surface

The peptides of the invention having affinity for a body surface comprise a body surface-binding peptide block (BSBPB) and a charged terminal peptide block at the N-terminus and/or C-terminus. Preferably, the peptides have a molecular weight of less than about 50,000 Daltons, more preferably, less than about 20,000 Daltons.

The body surface-binding peptide block comprises at least one body surface-binding peptide, which can be identified using the combinatorial methods described above. Non-limiting examples of body surface-binding peptides are peptides that bind to hair, skin, nails, teeth (e.g. tooth enamel and/or pellicle), gums, and the tissues of the oral cavity. In one embodiment, the body surface-binding peptide is a hair-binding (HBP) peptide. In another embodiment, the body surface-binding peptide is a skin-binding peptide (SBP). In another embodiment, the body surface-binding peptide is a nail-binding peptide (NBP). In another embodiment, the body surface-binding peptide is an oral cavity surface-binding peptide (OBP). In another embodiment, the body surface-binding peptide is a tooth-binding peptide (TBP). In a further embodiment, the tooth-binding peptide is a tooth-enamel binding peptide and/or a tooth pellicle-binding peptide. Examples of suitable hair, skin, nail, and tooth-binding peptides are given in Table A.

The body surface-binding peptide block of the peptides of the invention having affinity for a body surface may comprise a multiplicity of body surface-binding peptides (BSBPs) to enhance the interaction of the peptide with the body surface. Either multiple copies of the same body surface-binding peptide sequence or a combination of different body surface-binding peptide sequences may be coupled together, either directly or through a peptide spacer (S). For example, the body surface-binding peptide block may be a combination such as BSBP-BSBP-BSBP, wherein the sequences of the BSBPs are the same or different from each other. Additionally, the body surface-binding peptide block may be a combination such as BSBP-S-BSBP-S-BSBP, wherein the sequences of the BSBPs are the same or different from each other and S is a peptide spacer (as described below) linking two body surface-binding peptides. The sequences of the peptide spacers may be the same or different from each other. The body surface-binding peptide block may comprise up to about 50 body surface-binding peptides. Examples of body surface-binding peptide blocks comprising multiple hair-binding peptides are given as SEQ ID NOs:51-56, 69, and 70.

The charged terminal peptide block at the N-terminus (“nCPB+”) and/or the C-terminus (“cCPB+”) of the body surface-binding peptide block comprises at least 30 mole %, in addition at least 50 mole %, in addition at least 75 mole %, and further in addition 100 mole % of a charged amino acid selected from lysine, arginine, histidine, aspartic acid, glutamic acid, and combinations thereof. Preferably the charged terminal peptide block comprises at least 2, more preferably at least 3, and most preferably at least 4 of these charged amino acids. The remainder of the amino acids can be any natural amino acid, wherein glycine, alanine, proline, leucine, isoleucine, valine and phenylalanine are preferred. The charged peptide blocks are from 1 to about 50 amino acids in length.

In one embodiment, the charged terminal peptide block comprises positively charged amino acids selected from lysine, arginine, histidine and combinations thereof. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from non-charged amino acids such as glycine, alanine, proline, leucine, isoleucine, valine and phenylalanine.

In another embodiment, the charged terminal peptide block comprises negatively charged amino acids selected from aspartic acid, glutamic acid, and combinations thereof.

As described above, the body surface-binding peptide block comprising two or more body surface-binding peptides may be coupled together directly or via a peptide spacer (S). Additionally, the body surface-binding peptide block may be coupled to the charged terminal peptide blocks either directly or via a peptide spacer (Sn or Sc, designating spacers attached at the N-terminal or C-terminal end of the body surface-binding peptide block, respectively). These spacers serve to separate the peptide blocks to ensure that the binding affinity of the individual body surface-binding peptides is not adversely affected by the combination. The spacers may also provide other desirable properties such as hydrophilicity, hydrophobicity, or a means for cleaving the peptide sequences to facilitate removal of an attached benefit agent. The spacers are peptides comprising any natural amino acid and mixtures thereof. The peptide spacers may be from 1 to about 50 amino acids, preferably from 1 to about 20 amino acids in length. A spacer that confers hydrophilic properties comprises hydrophilic amino acids, including but not limited to, proline, serine, threonine, asparagine, glycine, glutamine, tyrosine, cysteine, lysine, arginine, histidine, aspartic acid, glutamic acid, and combinations thereof. A spacer comprising hydrophobic amino acids, including but not limited to, alanine, leucine, valine, glycine, proline, isoleucine, methionine, phenylalanine, tryptophan, and combinations thereof, confers hydrophobic properties to the peptide. The peptides of the invention may comprise a combination of different spacers. In addition, the peptide spacer may contain a specific enzyme cleavage site, such as the protease Caspase 3 site, given by SEQ ID NO:36, which allows for the enzymatic removal of the benefit agent from the body surface. Examples of suitable spacers include, but are not limited to, the sequences given by SEQ ID NOs:37-40.

Therefore, the peptides having affinity for a body surface have the general structure:

(nCPB^{±})ₓ-(Sn)-BSBPB-(Sc)-(cCPB^{±})_{y}
wherein:
(i) nCPB^{±} is an N-terminal charged peptide block, said N-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(ii) cCPB^{±} is a C-terminal charged peptide block, said C-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(iii) BSBPB is a body surface-binding peptide block comprising at least one body surface-binding peptide;
(iv) x and y are independently 0 or 1, provided that x and y may not both be 0; and
(v) Sn and Sc are optional peptide spacers comprised of 0 to about 20 amino acids.

Suitable examples of peptides having affinity for a body surface include, but are not limited to, sequences given as SEQ ID NOs:41-43, 58-63, 69, and 70.

The peptides when prepared by recombinant DNA and molecular cloning techniques, as described below and exemplified in Examples 1-3 herein, may further comprise a proline (P) residue at the N-terminus and optionally an aspartic acid (D) residue at the C-terminus. These additional residues result from the use of DP cleavage sites to separate the desired peptide sequence from peptide tags, used to promote inclusion body formation, and between tandem repeats of the peptide sequences.

### Production of Peptides

The peptides of the present invention having affinity for a body surface can be prepared using standard peptide synthesis methods, which are well known in the art (see for example, Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the peptides having affinity for a body surface may be produced in heterologous host cells, particularly in the cells of microbial hosts, as described by Huang et al. (U.S. Pat. No. 7,220,405), and as exemplified herein.

In addition, the peptide blocks (i.e., the body surface binding peptide block, the optional spacer, and the charged terminal peptide block) may be prepared separately using the methods described above and combined using coupling chemistries known in the art, such as carbodiimide coupling agents, diacid chlorides, diisocyanates and other bifunctional coupling reagents that are reactive to terminal amine and/or carboxylic acid terminal groups on the peptides (see for example, Hermanson, Bioconjugate Techniques, Academic Press, New York (1996)).

### Peptide-Based Body Surface Reagents

The peptide-based body surface reagents of the present invention are formed by coupling a peptide having affinity for a body surface, as described above, with at least one benefit agent (BA). The body surface-binding peptide part of the reagent binds strongly to the body surface, thus keeping the benefit agent attached to the body surface for a long lasting effect.

The peptide-based body surface reagents of the present invention are prepared by coupling a peptide having affinity to a body surface with a benefit agent, either directly or via an optional organic spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based body surface reagent may be prepared by mixing the peptide with the benefit agent and the optional spacer (if used) and allowing sufficient time for the interaction to occur. The unbound materials may be separated from the resulting peptide-based body surface reagent using methods known in the art, for example, chromatographic methods.

The peptide-based body surface reagents of the invention may also be prepared by covalently attaching the peptide having affinity for a body surface to a benefit agent, either directly or through an organic spacer. The benefit agent may be attached to any site on the peptide sequence. Any known peptide or protein conjugation chemistry may be used to form the peptide-based body surface reagents of the present invention. Conjugation chemistries are well-known in the art (see for example, Hermanson, Bioconjugate Techniques, Academic Press, New York (1996)). Suitable coupling agents include, but are not limited to, carbodiimide coupling agents, acid chlorides, isocyanates, epoxides, maleimides, and other functional coupling reagents that are reactive toward terminal amine and/or carboxylic acid groups, and sulfhydryl groups on the peptides. Additionally, it may be necessary to protect reactive amine or carboxylic acid groups on the peptide to produce the desired structure for the peptide-based body surface reagent. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, isocyanate, or aldehyde groups on the benefit agent for coupling to the peptide. These modifications may be done using routine chemistry such as oxidation, reduction, phosgenation, and the like, which is well known in the art.

It may also be desirable to couple the peptide of the invention to the benefit agent via an organic spacer (So). The organic spacer serves to separate the benefit agent from the peptide to ensure that the agent does not interfere with the binding of the peptide to the body surface. The spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, ethyl alkyl chains, propyl alkyl chains, hexyl alkyl chains, steryl alkyl chains, cetyl alkyl chains, and palmitoyl alkyl chains.

The spacer may be covalently attached to the peptide of the invention using any of the coupling chemistries described above. In order to facilitate incorporation of the spacer, a bifunctional coupling agent that contains two reactive groups available for coupling to the peptide and benefit agent may be used. Suitable bifunctional coupling agents are well known in the art and include, but are not limited to diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyanates, such as hexamethylenediisocyanate; bis oxiranes, such as 1,4 butanediyl diglycidyl ether; dicarboxylic acids, such as succinyldisalicylate; and the like. Heterobifunctional coupling agents, which contain two different reactive groups, may also be used.

It may also be desirable to have multiple peptides having affinity for a body surface coupled to the benefit agent to enhance the interaction between the peptide-based body surface reagent and the body surface. Either multiple copies of the same peptide or a combination of different peptides may be used. The number of copies of the peptide that can be attached to the benefit agent depends on the type of benefit agent used. When the benefit agent is a molecular species, such as a dye or non-particulate conditioning agent, from 1 to about 100, preferably from 1 to about 10 copies of the peptide may be attached to the benefit agent. When the benefit agent is a particle, such as a pigment, from 1 to about 10,000, preferably from 1 to about 2,000 copies of the peptide may be attached to the benefit agent. Additionally, multiple benefit agents may be coupled to the peptide at various sites along the peptide sequence (s). Multiple benefit agents may also be coupled to the organic spacer (s). From 1 to about 100, preferably from 1 to about 10 benefit agents may be attached to the peptide sequence or to the organic spacer.

Therefore, the peptide-based body surface reagents of the invention have the following general structure:

{(nCPB^{±})ₓ-(Sn)-BSBPB-(Sc)-(cCPB^{±})_{y}}_{z}-(So-BA_{S})ᵣ,

wherein:
(i) nCPB^{±} is an N-terminal charged peptide block, said N-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(ii) cCPB^{±} is a C-terminal charged peptide block, said C-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(iii) BSBPB is a body surface-binding peptide block comprising at least one body surface-binding peptide;
(iv) BA is a benefit agent;
(v) x and y are independently 0 or 1, provided that x and y may not both be 0;
(vi) z is 1 to about 10,000;
(vii) r and s are independently 1 to about 100;
(viii) So is an optional organic spacer; and
(ix) Sn and Sc are optional peptide spacers comprised of 0 to about 20 amino acids.

It should be understood that this structure is not meant to indicate that the benefit agent (BA) is coupled to the peptide only at the C-terminal end. As noted above, the benefit agent(s), optionally through the organic spacer (So) can be coupled to the peptide at any amino acid in the body surface-binding peptide block, the optional spacer blocks (Sc or Sn), or the charged terminal peptide blocks.

### Benefit Agents

The peptides of the invention having affinity for a body surface can be used in conjunction with a variety of benefit agents. The benefit agent by itself may or may not have affinity to a body surface. As used herein, a benefit agent “having affinity to a body surface” means that the benefit agent adsorbs onto the body surface and/or absorbs into the body surface. Suitable benefit agents include, but are not limited to, colorants, conditioning agents, sunscreen agents, oral benefit agents, and the like. Specific hair, skin, and oral benefit agents are discussed in detail below as examples of suitable benefit agents; however, it should be understood that the invention is not limited to these benefit agents. The peptides disclosed herein may used in combination with a wide range of benefit agents commonly used in the personal care industry.

Hair Benefit Agents

Any suitable hair benefit agent known in the art may be used in conjunction with the peptides of the invention. Suitable hair benefit agents include, but are not limited to, hair colorants and hair conditioning agents.

Hair colorants, as herein defined, are any dye, pigment, nanoparticle, and the like that may be used to change the color of hair. Hair coloring agents are well known in the art (see for example Green et al., WO 0107009, in particular page 42 line 1 to page 44 line 10, and CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Suitable hair coloring agents include, but are not limited to dyes, such as 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, 2-nitro-5-glyceryl methylaniline, 3-methylamino-4-nitrophenoxyethanol, 3-nitro-p-hydroxyethylaminophenol, hydroxyanthraquinoneaminopropylmethyl morpholinium methosulfate, Henna, HC Blue 1, HC Blue 2, HC Blue 26, HC Yellow 4, HC Red 3, HC Red 5, HC Red 7, HC Violet 1, HC Violet 2, HC Blue 7, HC Blue 10, HC Blue 12, HC Yellow 2, HC Yellow 6, HC Yellow 8, HC Yellow 12, HC Orange 2, HC Orange 3, HC Brown 2, D&C Yellow 1, D&C Yellow 3, D&C Blue 1, Disperse Blue 3, Disperse Violet 1, Disperse Orange, Disperse Violet 4, Disperse Black 9, Basic Orange 31, Basic Yellow 57, Basic Yellow 87, HC Yellow No. 9, Basic Blue 26, Basic Blue 7, Basic Blue 99, Basic Violet 14, Basic Violet 2, Basic Brown 16, Basic Brown 17, Basic Red 2, Basic Red 51, Acid Red 33, Brilliant Black 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, D&C Red No. 36, D&C Red No. 30, D&C Orange No. 17, Green 3 Lake, Ext. Yellow 7 Lake, Orange 4 Lake, and Red 28 Lake; the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of FD&C No. 40, of D&C Red Nos. 21, 22, 27, and 28, of FD&C Blue No. 1, of D&C Orange No. 5, of D&C Yellow No. 10, the zirconium lake of D&C Red No. 33; Cromophthal® Yellow 131AK (Ciba Specialty Chemicals), Sunfast® Magenta 122 (Sun Chemical) and Sunfast® Blue 15:3 (Sun Chemical), iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, titanium dioxide nanoparticles, zinc oxide, barium oxide, ultramarine blue, bismuth citrate, and white minerals such as hydroxyapatite, and Zircon (zirconium silicate), and carbon black particles.

Metallic and semiconductor nanoparticles may also be used as hair coloring agents due to their strong emission of light (Vic et al., U.S. Patent Application Publication No. 2004/0010864). The metallic nanoparticles include, but are not limited to, particles of gold, silver, platinum, palladium, iridium, rhodium, osmium, iron, copper, cobalt, and alloys composed of these metals. An “alloy” is herein defined as a homogeneous mixture of two or more metals. The “semiconductor nanoparticles” include, but are not limited to, particles of cadmium selenide, cadmium sulfide, silver sulfide, cadmium sulfide, zinc oxide, zinc sulfide, zinc selenide, lead sulfide, gallium arsenide, silicon, tin oxide, iron oxide, and indium phosphide. The nanoparticles are stabilized and made water-soluble by the use of a suitable organic coating or monolayer. As used herein, monolayer-protected nanoparticles are one type of stabilized nanoparticle. Methods for the preparation of stabilized, water-soluble metal and semiconductor nanoparticles are known in the art, and suitable examples are described by Huang et al. in co-pending and commonly owned U.S. Patent Application Publication No. 2004/0115345. The color of the nanoparticles depends on the size of the particles. Therefore, by controlling the size of the nanoparticles, different colors may be obtained.

Additionally, organic and inorganic nanoparticles, having attached or absorbed dye molecules, may be used as a hair coloring agent. For example, the hair coloring agent may be colored polymer nanoparticles. Exemplary polymer nanoparticles include, but are not limited to, microspheres comprised of materials such as polystyrene, polymethylmethacrylate, polyvinyltoluene, styrene/butadiene copolymer, and latex. For use in the invention, the colored microspheres have a diameter of about 10 nanometers to about 2 microns. The microspheres may be colored by coupling any suitable dye, such as those described above, to the microspheres. The dyes may be coupled to the surface of the microsphere or adsorbed within the porous structure of a porous microsphere. Suitable microspheres, including undyed and dyed microspheres that are functionalized to enable covalent attachment, are available from companies such as Bangs Laboratories (Fishers, Ind.).

Hair conditioning agents as herein defined are agents which improve the appearance, texture, and sheen of hair as well as increasing hair body or suppleness. Hair conditioning agents, include, but are not limited to, styling aids, hair straightening aids, hair strengthening aids, and volumizing agents, such as nanoparticles. Hair conditioning agents are well known in the art, see for example Green et al., supra (in particular page 51, line 26 to page 68, line 14) and are available commercially from various sources. Suitable examples of hair conditioning agents include, but are not limited to, cationic polymers, such as cationized guar gum, diallyl quaternary ammonium salt/acrylamide copolymers, quaternized polyvinylpyrrolidone and derivatives thereof, and various polyquaternium-compounds; long chain alkyl groups (i.e., C₈ to C₂₄); cationic surfactants, such as stearalkonium chloride, centrimonium chloride, and Sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and nanoparticles, such as silica nanoparticles and polymer nanoparticles. The preferred hair conditioning agents of the present invention contain amine or hydroxyl functional groups to facilitate coupling to the hair-binding peptides, as described below. Examples of preferred conditioning agents are octylamine (CAS No. 111-86-4), stearyl amine (CAS No. 124-30-1), behenyl alcohol (CAS No. 661-19-8, Cognis Corp., Cincinnati, Ohio), vinyl group terminated siloxanes, vinyl group terminated silicone (CAS No. 68083-19-2), vinyl group terminated methyl vinyl siloxanes, vinyl group terminated methyl vinyl silicone (CAS No. 68951-99-5), hydroxyl terminated siloxanes, hydroxyl terminated silicone (CAS No. 80801-30-5), amino-modified silicone derivatives, [(aminoethyl)amino]propyl hydroxyl dimethyl siloxanes, [(aminoethyl)amino]propyl hydroxyl dimethyl silicones, and alpha-tridecyl-omega-hydroxy-poly(oxy-1,2-ethanediyl) (CAS No. 24938-91-8).

Skin Benefit Agents

Any suitable skin benefit agent known in the art may be used in combination with the peptides of the invention. Suitable skin benefit agents include, but are not limited to skin colorants, skin conditioning agents, and sunscreen agents.

Skin colorants, as herein defined, are any dye, pigment, nanoparticle, and the like that may be used to change the color of skin. Any of the hair colorants described above may be used as a skin colorant. The preferred skin colorants for use in the method of the invention include the following dyes: eosin derivatives such as D&C Red No. 21 and halogenated fluorescein derivatives such as D&C Red No. 27, D&C Red Orange No. 5 in combination with D&C Red No. 21 and D&C Orange No. 10, and the pigments: titanium dioxide, titanium dioxide nanoparticles, zinc oxide, D&C Red No. 36 and D&C Orange No. 17, the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of D&C Red No. 27, of D&C Red No. 21, of FD&C Blue No. 1, iron oxides, manganese violet, chromium oxide, ultramarine blue, and carbon black. The coloring agent may also be a sunless tanning agent, such as dihydroxyacetone, that produces a tanned appearance on the skin without exposure to the sun.

Skin conditioning agents as herein defined include, but are not limited to, astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Any suitable known skin conditioning agent may be used in the method of the invention. Skin conditioning agents are well known in the art, see for example Green et al., supra (in particular page 44, line 11 to page 50, line 34) and are available commercially from various sources. Suitable examples of skin conditioning agents include, but are not limited to, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils and triglyceride esters. The preferred skin conditioning agents of the present invention are polysalicylates, propylene glycol (CAS No. 57-55-6, Dow Chemical, Midland, Mich.), glycerin (CAS No. 56-81-5, Proctor & Gamble Co., Cincinnati, Ohio), glycolic acid (CAS No. 79-14-1, DuPont Co., Wilmington, Del.), lactic acid (CAS No. 50-21-5, Alfa Aesar, Ward Hill, Mass.), malic acid (CAS No. 617-48-1, Alfa Aesar), citric acid (CAS No. 77-92-9, Alfa Aesar), tartaric acid (CAS N0.133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, Del.). Polysalicylates may be prepared by the method described by White et al. in U.S. Pat. No. 4,855,483. Glucaric acid may be synthesized using the method described by Merbouh et al. (Carbohydr. Res. 336:75-78 (2001). The 3-hydroxyvaleric acid may be prepared as described by Bramucci et al. in WO 02012530.

Sunscreen agents are substances that absorb, reflect, or scatter ultraviolet light at wavelengths from 290 to 400 nanometers. The sunscreen agents used in the invention may either be organic sunscreen agents or inorganic sunscreen agents. Organic sunscreen agents are herein defined as organic chemicals that absorb ultraviolet light of wavelengths between 290 and 400 nm. Organic sunscreen agents are well known in the art (see for example, Woddin et al., U.S. Pat. No. 5,219,558, in particular column 3 line 35 to column 4 line 23). Suitable examples include, but are not limited to, para-aminobenzoic acid (PABA), ethyl para-aminobenzoate, amyl para-aminobenzoate, octyl para-aminobenzoate, ethylhexyl dimethyl para-aminobenzoate (Padimate O), ethylene glycol salicylate, phenyl salicylate, octyl salicylate, benzyl salicylate, butylphenyl salicylate, homomethyl salicylate (Homosalate), ethylhexyl salicylate (Octisalate), TEA-salicylate (Trolamine salicylate), benzyl cinnamate, 2-ethoxyethyl para-methoxycinnamate (such as Parsol® available from Givaudan-Roure Co.), ethylhexyl methoxycinnamate (Octinoxate), octyl para-methoxycinnamate, glyceryl mono(2-ethylhexanoate) dipara-methoxycinnamate, isopropyl para-methoxycinnamate, urocanic acid, ethyl urocanate, hydroxymethoxybenzophenone (Benzophenone-3), hydroxymethoxybenzophenonesulfonic acid (Benzophenone-4) and salts thereof, dihydroxymethoxybenzophenone (Benzophenone-8), sodium dihydroxymethoxybenzophenonedisulfonate, dihydroxybenzophenone, tetrahydroxybenzophenone, 4-tert-butyl-4′-methoxydibenzoylmethane (Avobenzone), phenylbenzimidazole sulfonic acid (Ensulizole), 2,4,6-trianilino-p-(carbo-2′-ethylhexyl-1′-oxy)-1,3,5-triazine, Octocrylene, menthyl anthranilate (Meradimate), and 2-(2-hydroxy-5-methylphenyl)benzotriazole.

Inorganic UV sunscreen agents are typically inorganic pigments and metal oxides including, but not limited to, titanium dioxide (such as SunSmart available from Cognis Co.), zinc oxide, and iron oxide. A preferred sunscreen agent is titanium dioxide nanoparticles. Suitable titanium dioxide nanoparticles are described in U.S. Pat. Nos. 5,451,390; 5,672,330; and 5,762,914. Titanium dioxide P25 is an example of a suitable commercial product available from Degussa (Parsippany, N.J.). Other commercial suppliers of titanium dioxide nanoparticles include Kemira (Helsinki, Finland), Sachtleben (Duisburg, Germany) and Tayca (Osaka, Japan).

The titanium dioxide nanoparticles typically have an average particle size diameter of less than 100 nanometers (nm) as determined by dynamic light scattering which measures the particle size distribution of particles in liquid suspension. The particles are typically agglomerates which may range from about 3 nm to about 6000 nm. Any process known in the art can be used to prepare such particles. The process may involve vapor phase oxidation of titanium halides or solution precipitation from soluble titanium complexes, provided that titanium dioxide nanoparticles are produced.

A preferred process to prepare titanium dioxide nanoparticles is by injecting oxygen and titanium halide, preferably titanium tetrachloride, into a high-temperature reaction zone, typically ranging from 400 to 2000 degrees centigrade. Under the high temperature conditions present in the reaction zone, nanoparticles of titanium dioxide are formed having high surface area and a narrow size distribution. The energy source in the reactor may be any heating source such as a plasma torch.

Oral Benefit Agents

Any suitable oral benefit agent known in the art (see for example White et al., U.S. Pat. No. 6,740,311; Lawler et al., U.S. Pat. No. 6,706,256; and Fuglsang et al., U.S. Pat. No. 6,264,925, may be used in combination with the peptides of the invention. Suitable oral benefit agents include, but are not limited to, white colorants, whitening agents, enzymes, anti-plaque agents, anti-staining agents, anti-microbial agents, anti-caries agents, flavoring agents, coolants, and salivating agents.

Suitable white colorants which may be used in combination with the peptides and peptide-based body surface reagents disclosed herein to whiten teeth, include, but are not limited to, white pigments such as titanium dioxide, titanium dioxide nanoparticles; and white minerals such as hydroxyapatite, and Zircon (zirconium silicate). Suitable enzymes may be naturally occurring or recombinant enzymes including, but not limited to, oxidases, peroxidases, perhydrolases, proteases, lipases, glycosidases, esterases, and polysaccharide hydrolases. Anti-plaque agents include, but are not limited to, fluoride ion sources and anti-microbial agents. Suitable anti-microbial agents include, but are not limited to, anti-microbial peptides such as those described by Haynie in U.S. Pat. No. 5,847,047, magainins, and cecropins; microbiocides such as triclosan, chlorhexidine, quaternary ammonium compounds, chloroxylenol, chloroxyethanol, phthalic acid and its salts, and thymol. Suitable flavoring agents include, but are not limited to, oil of wintergreen, oil of peppermint, oil of spearmint, menthol, methyl salicylate, eucalyptol, and vanillin.

### Personal Care Compositions

The peptides having affinity for a body surface and the peptide-based body surface reagents of the invention are used in personal care compositions. The peptides can be used as sealants to attach a benefit agent to a body surface. The peptide-based body surface reagents can be used to deliver the coupled benefit agent to a body surface or can be used as sealants. Additionally, the peptides can be used in combination with peptide-based body surface reagents to seal the peptide-based body surface reagent to the body surface.

Suitable personal care compositions include, but are not limited to, hair care, skin care, nail care, and oral care compositions.

Hair Care Compositions

The peptides having affinity for a body surface and the peptide-based body surface reagents disclosed herein can be used in various hair care compositions. For use in hair care compositions, the peptide and the peptide-based body surface reagent comprise a hair-binding peptide block and therefore, have an affinity for hair.

Hair care compositions are herein defined as compositions for the treatment of hair including, but not limited to, shampoos, conditioners, rinses, lotions, aerosols, gels, mousses, and hair colorants. The peptide having affinity for hair and/or the peptide-based body surface reagent is used in the composition at a concentration of about 0.01% to about 10%, preferably about 0.01% to about 5% by weight relative to the total weight of the composition. This proportion may vary as a function of the type of hair care composition. The hair care composition may further comprise at least one hair benefit agent. The concentration of the peptide and/or peptide-based body surface reagent in relation to the concentration of the benefit agent may need to be optimized for best results. Suitable benefit agents are described above. Additionally, a mixture of different peptides and/or peptide-based body surface reagents may be used in the composition. The peptides and/or peptide-based body surface reagents in the mixture need to be chosen so that there is no interaction between them that mitigates the beneficial effect. Suitable mixtures of peptides and/or peptide-based body surface reagents may be determined by one skilled in the art using routine experimentation. If a mixture of peptides and/or peptide-based body surface reagents is used in the composition, the total concentration of the peptides and/or peptide-based body surface reagents is about 0.01% to about 10% by weight relative to the total weight of the composition.

The hair care composition may further comprise a cosmetically acceptable medium for hair care compositions, examples of which are described by Philippe et al. in U.S. Pat. No. 6,280,747, and by Omura et al. in U.S. Pat. No. 6,139,851 and by Cannell et al. in U.S. Pat. No. 6,013,250. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including, but not limited to, antioxidants; preserving agents; fillers; surfactants, UVA and/or UVB sunscreens; fragrances; thickeners; wetting agents; anionic, nonionic or amphoteric polymers; and dyes or pigments.

Skin Care Compositions

The peptides having affinity for a body surface and the peptide-based body surface reagents disclosed herein can be used in various skin care compositions. For use in skin care compositions, the peptide and the peptide-based body surface reagent comprise a skin-binding peptide block and therefore, have an affinity for skin.

Skin care compositions are herein defined as compositions for the treatment of skin including, but not limited to, skin care, skin cleansing, make-up, and anti-wrinkle products. The peptide having affinity for skin and/or the peptide-based body surface reagent is used in the composition at a concentration of about 0.01% to about 10%, preferably about 0.01% to about 5% by weight relative to the total weight of the composition. This proportion may vary as a function of the type of skin care composition. The skin care composition may further comprise at least one skin benefit agent, examples of which are describe above. The concentration of the peptide and/or peptide-based body surface reagent in relation to the concentration of the benefit agent may need to be optimized for best results. Additionally, a mixture of different peptides and/or peptide-based body surface reagents may be used in the composition. The peptides and/or peptide-based body surface reagents in the mixture need to be chosen so that there is no interaction between the them that mitigates the beneficial effect. Suitable mixtures of peptides and/or peptide-based body surface reagents may be determined by one skilled in the art using routine experimentation. If a mixture of peptides and/or peptide-based body surface reagents is used in the composition, the total concentration of the peptides and/or peptide-based body surface reagents is about 0.01% to about 10% by weight relative to the total weight of the composition.

The skin care composition may further comprise a cosmetically acceptable medium for skin care compositions, examples of which are described by Philippe et al. supra. For example, the cosmetically acceptable medium may be an anhydrous composition containing a fatty substance in a proportion generally of from about 10 to about 90% by weight relative to the total weight of the composition, where the fatty phase contains at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes or pigments.

Oral Care Compositions

The peptides having affinity for a body surface and the peptide-based body surface reagents disclosed herein can be used in various oral care compositions. For use in oral care compositions, the peptide and the peptide-based body surface reagent comprise a peptide block having affinity for teeth, gums, cheeks, tongue, or other surfaces in the oral cavity.

Oral care compositions are herein defined as compositions for the treatment of teeth, gum, cheeks, tongue, or other surfaces in the oral cavity. The oral care compositions may have any suitable physical form, such as powder, paste, gel, liquid, ointment, or tablet. Exemplary oral care compositions include, but are not limited to, toothpaste, dental cream, gel or tooth powder, mouth wash, breath freshener, and dental floss. The peptide having affinity for a surface in the oral cavity and/or the peptide-based body surface reagent is used in the composition at a concentration of about 0.01% to about 10%, preferably about 0.01% to about 5% by weight relative to the total weight of the composition. This proportion may vary as a function of the type of oral care composition. The oral care composition may further comprise at least one oral benefit agent, examples of which are described above. The concentration of the peptide and/or peptide-based body surface reagent in relation to the concentration of the benefit agent may need to be optimized for best results. Additionally, a mixture of different peptides and/or peptide-based body surface reagents may be used in the composition. The peptides and/or peptide-based body surface reagents in the mixture need to be chosen so that there is no interaction between them that mitigates the beneficial effect. Suitable mixtures of peptides and/or peptide-based body surface reagents may be determined by one skilled in the art using routine experimentation. If a mixture of peptides and/or peptide-based body surface reagents is used in the composition, the total concentration of the peptides and/or peptide-based body surface reagents is about 0.01% to about 10% by weight relative to the total weight of the composition.

The oral care composition may further comprise a cosmetically acceptable medium for oral care compositions, examples of which are described by White et al., supra; Lawler et al., supra; and Fuglsang et al., supra. For example, the oral care composition may contain one or more of the following: abrasives, surfactants, chelating agents, fluoride sources, thickening agents, buffering agents, solvents, humectants, carriers, bulking agents, and additional oral benefit agents, as given above.

Nail Polish Compositions

The peptides having affinity for a body surface and the peptide-based body surface reagents disclosed herein can be used in various nail polish compositions. For use in nail polish compositions, the peptide and the peptide-based body surface reagent comprise a nail-binding peptide block and therefore, have an affinity for nails.

Nail polish compositions are herein defined as compositions for the treatment and coloring of fingernails and toenails. The peptide having affinity for nails and/or the peptide-based body surface reagent is used in the composition at a concentration of about 0.01% to about 10%, preferably about 0.01% to about 5% by weight relative to the total weight of the composition. This proportion may vary as a function of the type of nail polish composition. The composition may further comprise at least one oral benefit agent. The concentration of the peptide and/or peptide-based body surface reagent in relation to the concentration of the benefit agent may need to be optimized for best results. Additionally, a mixture of different peptides and/or peptide-based body surface reagents may be used in the composition. The peptides and/or peptide-based body surface reagents in the mixture need to be chosen so that there is no interaction between the them that mitigates the beneficial effect. Suitable mixtures of peptides and/or peptide-based body surface reagents may be determined by one skilled in the art using routine experimentation. If a mixture of peptides and/or peptide-based body surface reagents is used in the composition, the total concentration of the peptides and/or peptide-based body surface reagents is about 0.01% to about 10% by weight relative to the total weight of the composition.

The nail polish composition may further comprise a cosmetically acceptable medium for nail polish compositions, examples of which are described by Philippe et al. supra. The nail polish composition typically contains a solvent and a film forming substance, such as cellulose derivatives, polyvinyl derivatives, acrylic polymers or copolymers, vinyl copolymers and polyester polymers. Additionally, the nail composition may contain a plasticizer, such as tricresyl phosphate, benzyl benzoate, tributyl phosphate, butyl acetyl ricinoleate, triethyl citrate, tributyl acetyl citrate, dibutyl phthalate or camphor.

### Methods for Applying a Benefit Agent to a Body Surface

The peptides having affinity for a body surface and the peptide-based body surface reagents of the invention may be used to apply a benefit agent to a body surface in various ways, as described below.

In one embodiment, the peptide-based body surface reagents of the invention are used to deliver a benefit agent to a body surface. In this embodiment, a composition comprising a peptide-based body surface reagent is applied to a body surface for a time sufficient for the peptide-based body surface reagent to bind to the body surface, preferably for at least about 5 seconds to 60 minutes. After this time, the composition may be left on the body surface or be rinsed from the body surface. The compositions of the present invention may be applied to the body surface by various means, including, but not limited to spraying, brushing, and applying by hand.

In another embodiment, the peptides having affinity for a body surface or the peptide-based body surface reagents of the invention are used as a sealant to enhance the durability of common benefit agents, for example, colorants or conditioning agents. The peptide-based body surface reagent may comprise the same benefit agent or a different benefit agent than that being sealed to the body surface. For example, the benefit agent being sealed to the body surface may be a hair dye and the peptide-based body surface reagent may comprise a hair conditioning agent. Additionally, the benefit agent being sealed to the body surface may be a hair dye and the peptide-based body surface reagent may comprise the same hair dye or another hair colorant. The benefit agent may also be applied in the form of a peptide-based body surface reagent and sealed to the body surface using a peptide having affinity for the body surface or another peptide-based body surface reagent. All these and other possible combinations are within the scope of the invention. The benefit agent may be applied to the body surface from any suitable personal care composition, as described above.

In another embodiment, a benefit agent is applied to the body surface for a time sufficient for the benefit agent to bind to the body surface, typically from about 5 seconds to about 60 minutes. Optionally, the body surface may be rinsed to remove the benefit agent that has not bound. Then, a composition comprising a peptide having affinity of the body surface and/or a peptide-based body surface reagent is applied to the body surface for a time sufficient for the peptide to bind to the body surface, typically from about 5 seconds to about 60 minutes. The composition may be rinsed from the body surface or left on the body surface.

In another embodiment, the composition comprising a peptide having affinity for a body surface and/or a peptide-based body surface reagent is applied to the body surface for a time sufficient for the peptide to bind to the body surface, typically from about 5 seconds to about 60 minutes. Optionally, the body surface may be rinsed to remove the composition that has not bound. Then, a benefit agent is applied to the body surface for a time sufficient for the benefit agent to bind to the body surface, typically from about 5 seconds to about 60 minutes. The unbound benefit agent may be rinsed from the body surface or left on the body surface.

In another embodiment, the benefit agent and the composition comprising a peptide having affinity for a body surface or a peptide-based peptide reagent are applied to the body surface concomitantly for a time sufficient for the benefit agent and the peptide or the peptide-based peptide reagent to bind to the body surface, typically from about 5 seconds to about 60 minutes. Optionally, the body surface may be rinsed to remove the unbound benefit agent and the composition.

In another embodiment, the benefit agent is provided as part of the composition comprising a peptide having affinity for a body surface and/or a peptide-based body surface reagent. In this embodiment, the composition is applied to the body surface for a time sufficient for the benefit agent and the peptide to bind to the body surface, typically from about 5 seconds to about 60 minutes. The composition may be rinsed from the body surface or left on the body surface.

In any of the methods described above, the composition comprising a peptide having affinity for a body surface and/or a peptide-based body surface reagent may be optionally reapplied to the body surface after the application of the benefit agent and the composition in order to further enhance the durability of the benefit agent.

Additionally, in any of the methods described above, a composition comprising a polymeric sealant may be optionally applied to the body surface after the application of the benefit agent and the composition comprising a peptide having affinity for a body surface and/or a peptide-based-body surface reagent in order to further enhance the durability of the benefit agent. The composition comprising the polymeric sealant may be an aqueous solution or a personal care composition comprising the polymeric sealant. Typically, the polymeric sealant is present in the composition at a concentration of about 0.25% to about 10% by weight based on the total weight of the composition. Polymeric sealants are well know in the art of personal care products and include, but are not limited to, poly(allylamine), polyethyleneimine, acrylates, acrylate copolymers, polyurethanes, carbomers, methicones, amodimethicones, polyethylenene glycol, beeswax, siloxanes, and the like. The choice of polymeric sealant depends on the particular benefit agent and the body surface-binding peptide used. The optimum polymeric sealant may be readily determined by one skilled in the art using routine experimentation.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “sec” means second(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mol” means mole(s), mmol” means millimole(s), “μmol” means micromole(s), “pmol” means picomole(s), “g” means gram(s), “μg” means microgram(s), “mg” means milligram(s), “wt %” means percent by weight, “vol %” means percent by volume, “TBS” means Tris-buffered saline, “TBST-X” means Tris-buffered saline containing TWEEN® 20 where “X” is the weight percent of TWEEN® 20, “HPLC” means high performance liquid chromatography, “OD₆₀₀” means the optical density measured at a wavelength of 600 nm, “rpm” means revolutions per minute, “atm” means atmosphere(s), “kPa” means kilopascals, “SLPM” means standard liter per minute, “psi” means pounds per square inch, “RCF” means relative centrifugal field, and “nd” means not determined.

### Examples 1-3

### Biological Production of Peptides Having Affinity for Hair Comprising a Hair-Binding Peptide Block and a Charged Terminal Peptide Block

The purpose of these Examples was to prepare peptides comprising a hair-binding peptide block and a charged terminal peptide block using recombinant DNA and molecular cloning techniques. The peptides were expressed in E. coli as inclusion bodies. Additional amino acid sequences (i.e., peptide tags) were fused to the peptide sequences in order to promote inclusion body formation. Acid-labile Asp-Pro (DP) sequences were placed between the peptide tag and the peptide sequences and between tandem repeats of the peptide sequences.

Of note is that the peptides or the peptide-based body surface reagents of the invention wherein the peptide having affinity for a body surface further comprises a proline residue on the N-terminal end and optionally an aspartic acid residue on the C-terminal end.

### Construction of Production Strains

The sequences of the biologically produced peptides are given in Table 1. DNA sequences were designed to encode these peptides using favorable codons for E. coli and to avoid sequence repeats and mRNA secondary structure. The gene DNA sequence was designed by DNA 2.0, Inc. (Menlo Park, Calif.) using proprietary software which is described by Gustafsson et al. (Trends in Biotechnol. 22(7):346-355 (2004)). In each case the sequence encoding the amino acid sequence was followed by two termination codons and a recognition site for endonuclease Ascl. The GS amino acid sequence at the N-terminus was encoded by a recognition site for endonuclease BamHI (GGA/TCC). The DNA coding sequences are given in Table 1.

TABLE 1

Sequences of Peptides Comprising a Hair-Binding Peptide
Block and a Charged Terminal Peptide Block

| | | | Peptide | DNA |
|---|---|---|---|---|
| | | | SEQ ID | SEQ ID |
| Example | Peptide | Expressed Peptide Sequence | NO: | NO: |
| 1 | HC 71E | GSDP-TPPELLHGAPRS-KRKRK-D | 44 | 45 |
| | | P-TPPELLHGAPRS-KRKRK-D | | |
| | | P-TPPELLHGAPRS-KRKRK-D | | |
| | | P-TPPELLHGAPRS-KRKRK-D | | |
| 2 | HC 74E | GSDP-EQISGSLVAAPW-KRKRK-D | 46 | 47 |
| | | P-EQISGSLVAAPW-KRKRK-D | | |
| | | P-EQISGSLVAAPW-KRKRK-D | | |
| | | P-EQISGSLVAAPW-KRKRK-D | | |
| 3 | HC 81E | GSDP-NTSQLST-GGG- | 48 | 49 |
| | | GHGHQKQHGLGHGHKHGHGHGH | | |
| | | GHGK | | |

Genes were assembled from synthetic oligonucleotides and cloned into a standard plasmid cloning vector by DNA 2.0, Inc. Sequences were verified by DNA sequencing by DNA 2.0, Inc.

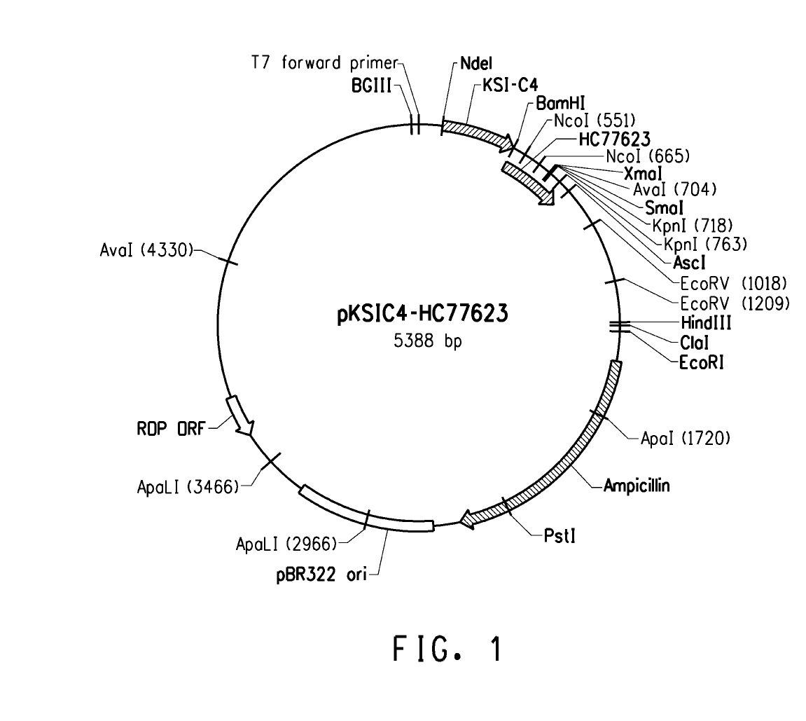

The synthetic genes were excised from the cloning vector with the endonuclease restriction enzymes BamHI and AscI and ligated into an expression vector using standard recombinant DNA methods. The vector pKSIC4-HC77623 was derived from the commercially available vector pDEST17 (Invitrogen, Carlsbad, Calif.). It includes sequences derived from the commercially available vector pET31b (Novagen, Madison, Wis.) that encode a fragment of the enzyme ketosteroid isomerase (KSI). The KSI fragment was included as a fusion partner to promote partition of the peptides into insoluble inclusion bodies in E. coli. The KSI-encoding sequence from pET31b was modified using standard mutagenesis procedures (QuickChange II, Stratagene, La Jolla, Calif.) to include three additional Cys codons, in addition to the one Cys codon found in the wild type KSI sequence. The plasmid pKSIC4—HC77623, given by SEQ ID NO:50 and shown in FIG. 1, was constructed using standard recombinant DNA methods, which are well known to those skilled in the art.

The DNA sequences encoding the desired peptides (Table 1) were inserted into pKSIC4—HC77623 by substituting for sequences in the vector between the BamHI and AscI sites. Plasmid DNA containing the peptide encoding sequences and vector DNA were digested with endonuclease restriction enzymes BamHI and Ascl, then the peptide-encoding sequences and vector DNA were mixed and ligated by phage T4 DNA ligase using standard DNA cloning procedures, which are well known to those skilled in the art. Correct constructs, in which the sequences encoding the desired peptide were respectively inserted into pKSIC4-HC77623, were identified by restriction analysis and verified by DNA sequencing, using standard methods.

In these constructs, the sequences encoding the peptides of interest were substituted for those encoding HC77623. These sequences were operably linked to the bacteriophage T7 gene 10 promoter and expressed as a fusion protein, fused with the variant KSI partner.

To test the expression of the peptides, the expression plasmids were transformed into the BL21-Al E. coli strain (Invitrogen, catalog no. C6070-03). To produce the recombinant fusion peptides, 50 mL of LB-ampicillin broth (10 g/L bacto-tryptone, 5 g/L bacto-yeast extract, 10 g/L NaCl, 100 mg/L ampicillin, pH 7.0) was inoculated with the transformed bacteria and the culture was shaken at 37° C. until the OD₆₀₀ reached 0.6. The expression was induced by adding 0.5 mL of 20 wt % L-arabinose to the culture and shaking was continued for another 4 h. Analysis of the cell protein by polyacrylamide gel electrophoresis demonstrated the production of the fusion peptides.

### Fermentation:

The recombinant E. coli strains, described above, were grown in a 6-L fermentation, which was run in batch mode initially, and then in fed-batch mode. The composition of the fermentation medium is given in Table 2. The pH of the fermentation medium was 6.7. The fermentation medium was sterilized by autoclaving, after which the following sterilized components were added: thiamine hydrochloride (4.5 mg/L), glucose (22.1 g/L), trace elements, see Table 3 (10 mL/L), ampicillin (100 mg/L), and inoculum (seed) (125 mL). The pH was adjusted as needed using ammonium hydroxide (20 vol %) or phosphoric acid (20 vol %). The added components were sterilized either by autoclaving or filtration.

TABLE 2
Composition of Fermentation Medium

| Component | Concentration | |
|---|---|---|
| KH₂PO₄ | 9 | g/L |
| (NH₄)₂HPO₄ | 4 | g/L |
| MgSO₄•7H₂O | 1.2 | g/L |
| Citric Acid | 1.7 | g/L |
| Yeast extract | 5.0 | g/L |
| Mazu DF 204 Antifoam | 0.1 | mL/L |

TABLE 3
Trace Elements

| Component | Concentration, mg/L |
|---|---|
| EDTA | 840 |
| CoCl₂•H₂O | 250 |
| MnCl₂•4H₂O | 1500 |
| CuCl₂•2H₂O | 150 |
| H₃BO₃ | 300 |
| Na₂MoO₄•2H₂O | 250 |
| Zn(CH₃COO)₂•H₂O | 1300 |
| Ferric citrate | 10000 |

The operating conditions for the fermentations are summarized in Table 4. The initial concentration of glucose was 22.1 g/L. When the initial residual glucose was depleted, a pre-scheduled, exponential glucose feed was initiated starting the fed-batch phase of the fermentation run. The glucose feed (see Tables 5 and 6) contained 500 g/L of glucose and was supplemented with 5 g/L of yeast extract. The components of the feed medium were sterilized either by autoclaving or filtration. The goal was to sustain a specific growth rate of 0.13 h⁻¹, assuming a yield coefficient (biomass to glucose) of 0.25 g/g, and to maintain the acetic acid levels in the fermentation vessel at very low values (i.e., less than 0.2 g/L). The glucose feed continued until the end of the run. Induction was initiated with a bolus of 2 g/L of L-arabinose at the selected time (i.e., 15 h of elapsed fermentation time). A bolus to deliver 5 g of yeast extract per liter of fermentation broth was added to the fermentation vessel at the following times: 1 h prior to induction, at induction time, and 1 h after induction time. The fermentation run was terminated after 19.97 h of elapsed fermentation time, and 4.97 h after the induction time.

TABLE 4
Fermentation Operating Conditions

| Condition | Initial | | Minimum | | Maximum | |
|---|---|---|---|---|---|---|
| Stirring | 220 | rpm | 220 | rpm | 1200 | rpm |
| Air Flow | 3 | SLPM | 3 | SLPM | 30 | SLPM |
| Temperature | 37° | C. | 37° | C. | 37° | C. |
| pH | 6.7 | | 6.7 | | 6.7 | |
| Pressure | 0.500 | atm | 0.500 | atm | 0.500 | atm |
| | (50.7 | kPa) | (50.7 | kPa) | (50.7 | kPa) |
| Dissolved O₂* | 20% | | 20% | | 20% | |

*Cascade stirrer, then air flow.

TABLE 5
Composition of Feed Medium

| Component | Concentration | |
|---|---|---|
| MgSO₄•7H₂O | 2.0 | g/L |
| Glucose | 500 | g/L |
| Ampicillin | 150 | mg/L |
| (NH₄)₂HPO₄ | 4 | g/L |
| KH₂PO₄ | 9 | g/L |
| Yeast extract | 5.0 | g/L |
| Trace Elements - Feed (Table 6) | 10 | mL/L |

TABLE 6
Trace Elements - Feed

| Component | Concentration, mg/L |
|---|---|
| EDTA | 1300 |
| CoCl₂•H₂O | 400 |
| MnCl₂•4H₂O | 2350 |
| CuCl₂•2H₂O | 250 |
| H₃BO₃ | 500 |
| Na₂MoO₄•2H₂O | 400 |
| Zn(CH₃COO)₂•H₂O | 1600 |
| Ferric citrate | 4000 |

### Isolation and Purification of Peptides:

After completion of the fermentation run, the entire fermentation broth was passed three times through an APV model 2000 Gaulin type homogenizer at 12,000 psi (82,700 kPa). The broth was cooled to below 5° C. prior to each homogenization. The homogenized broth was immediately processed through a Westfalia WhisperFuge™ (Westfalia Separator Inc., Northvale, N.J.) stacked disc centrifuge at 700 mL/min and 12,000 RCF to separate inclusion bodies from suspended cell debris and dissolved impurities. The recovered paste was resuspended at 15 g/L (dry basis) in water and the pH was adjusted to a value between 8.0 and 10.0 using Na₂CO₃/NaOH buffer. The pH was chosen to help remove cell debris from the inclusion bodies without dissolving the inclusion body proteins. The suspension was passed through the APV 2000 Gaulin type homogenizer at 12,000 psi (82,700 kPa) for a single pass to provide rigorous mixing. The homogenized high pH suspension was immediately processed in a Westfalia WhisperFuge™ stacked disc centrifuge at 700 mL/min and 12,000 RCF to separate the washed inclusion bodies from suspended cell debris and dissolved impurities. The recovered paste was resuspended at 15 gm/L (dry basis) in pure water. The suspension was passed through the APV 2000 Gaulin type homogenizer at 12,000 psi (82,700 kPa) for a single pass to provide rigorous washing. The homogenized suspension was immediately processed in a Westfalia WhisperFuge™ stacked disc centrifuge at 700 mL/min and 12,000 RCF to separate the washed inclusion bodies from residual suspended cell debris and NaOH.

The recovered paste was resuspended in pure water at 25 g/L (dry basis) and the pH of the mixture was adjusted to 2.2 using HCl. The acidified suspension was heated to 70° C. for 5 to 14 h to complete cleavage of the DP site separating the fusion peptide from the product peptide without damaging the target peptide. The product slurry was adjusted to pH 5.1 (note: the pH used here may vary depending on the solubility of the peptide being recovered) using NaOH and then was cooled to 5° C. and held for 12 h. The mixture was centrifuged at 9000 RCF for 30 min and the supernatant was decanted. The supernatant was then filtered with a 0.2 μm membrane. For some low solubility peptides, multiple washes of the pellet were required to increase peptide recovery.

The filtered product was collected and concentrated by vacuum evaporation by a factor of 2:1 before lyophilization. In the case of HC 81, the peptide was purified by reverse phase chromatography using an acetonitrile gradient (0 to 100%) in water with 0.1 v/v % trifluoroacetic acid and a C18 column. The fraction containing the peptide was collected, concentrated by evaporation and lyophilized. Spectrophotometric detection at 220 and 278 nm was used to monitor and track elution of the product peptide.

As described above, the peptides produced are cleaved at the DP site using acid, so that the resulting peptides having sequences begin with proline and end with aspartic acid. Therefore, the sequences of the three final peptide products HC 71, HC 74, and HC 81 are: P(from DP cleavage site)-TPPELLHGAPRS(hair-binding peptide block)-KRKRK(charged terminal peptide block)-D(from DP cleavage site), given as SEQ ID NO:41; P(from DP cleavage site)-EQISGSLVAAPW(hair-binding peptide block)-KRKRK(charged terminal peptide block)-D(from DP cleavage site), given as SEQ ID NO:42; and P(from DP cleavage site)—NTSQLST(hair-binding peptide block)-GGG(peptide spacer)-GHGHQKQHGLGHGHKHGHGH GHGHGK(charged terminal peptide block), given as SEQ ID NO:43, respectively.

In one embodiment, the peptides or the peptide-based body surface reagents of the invention wherein the peptide having affinity for a body surface further comprises a proline residue on the N-terminal end and optionally an aspartic acid residue on the C-terminal end.

### Examples 4-7

### Coloring Hair Using Peptides Having Affinity for Hair as a Sealant

The purpose of these Examples was to demonstrate the coloring of hair using a hair dye in combination with a peptide comprising a hair-binding peptide block and a charged terminal peptide block as a sealant. The peptides used in this Example were prepared as described in Examples 1-3. The color retention was quantified using a spectrophotometric measurement technique.

A peptide given as SEQ ID NO:41, SEQ ID NO:42, or SEQ ID NO:43 (38 mg), produced via fermentation as described in Examples 1-3, was added to 15 g of an aqueous 0.50 wt % solution of Acid Red 33 (Abbey Color, Philadelphia, Pa.) and the solution was allowed to stir for 1 h. A natural white hair tress (1 cm wide, potted, International Hair Importers & Products Inc., Bellerose, N.Y.) was inserted into a 15 mm×125 mm test tube and 13 mL of the peptide/dye mixture was injected into the test tube. The hair tress was immersed in contact with the colorant solution for 30 min and the colorant solution was agitated using a magnetic stirrer. The tress was removed, allowed to drip dry, and then air dried for 30 min.

The hair tress was then subjected to a water rinse using copious amounts of deionized water, followed by a shampoo treatment with embrocation for 30 seconds. The shampoo treatment involved the application of Pantene Pro-V Sheer Volume shampoo (Proctor & Gamble, Cincinnati, Ohio) to the hair as follows. A quarter-sized drop of the shampoo was distributed evenly over the hair tress and then was massaged aggressively into the hair for 30 sec, after which the hair tress was rinsed with water to remove the shampoo. The hair tress was then dried at room temperature. The entire procedure described above was repeated without the addition of the hair-binding peptide to serve as a control.

The color intensity after the shampoo treatment was measured using an X-Rite® SP78™ Sphere Spectrophotometer (X-Rite, Inc., Grandville, Mich.), by placing the colored hair sample into the photosensor and calculating L*, a* and b* parameters representing the photometer response. An initial baseline L* value was measured for the uncolored hair and all measurements were the average of three individual determinations. Delta E values were calculated using equation 1 below:

Delta E=((L*₁−L*₂)²+(a₁−a₂)²+(b₁−b₂)²)^{1/2} (1)

where L*=the lightness variable and a* and b* are the chromaticity coordinates of CIELAB colorspace as defined by the International Commission of Illumination (CIE) (Minolta, Precise Color Communication—Color Control From Feeling to Instrumentation, Minolta Camera Co., 1996). Larger Delta E value are indicative of better color retention. The results are summarized in Table 7.

TABLE 7
Results of Color Retention After Shampoo Treatment

| | | | Peptide | |
|---|---|---|---|---|
| Example | Peptide | SEQ ID NO: | Conc. wt. % | Delta E |
| 4 | HC 71 | 41 | 0.25 | 25.80 |
| 5 | HC 74 | 42 | 0.25 | 28.68 |
| 6 | HC 81 | 43 | 0.25 | 31.57 |
| 7 | None | — | 0 | 19.63 |
| Comparative | | | | |

As can be seen from the results in Table 7, the use of the hair-binding peptides (Examples 4-6) as a sealant for the dye provided significantly better hair color retention, as measured by the Delta E values, than the use of the dye alone (Comparative Example 7).

### Examples 8-10

### Coloring Hair Using Peptides Having Affinity for Hair as a Sealant

The purpose of these Examples was to demonstrate the coloring of hair using a hair dye in combination with a peptide comprising a hair-binding peptide block and a charged terminal peptide block as a sealant. The peptides used in this Example were prepared as described in Examples 1 and 2. The color retention was quantified using a spectrophotometric measurement technique.

A peptide given as SEQ ID NO:41 or SEQ ID NO:42 (38 mg), produced via fermentation as described in Examples 1 and 2, was added to 15 g of an aqueous 0.25 wt % solution of Basic Red 2 (Abbey Color, Philadelphia, Pa.) and the solution was allowed to stir for 1 h. A natural white hair tress (1 cm wide, potted, International Hair Importers & Products Inc., Bellerose, N.Y.) was inserted into a 15 mm×125 mm test tube and 13 mL of the peptide/dye mixture was injected into the test tube. The hair tress was immersed in contact with the colorant solution for 60 min and the colorant solution was agitated using a magnetic stirrer. The tress was removed, allowed to drip dry, and then air dried for 30 min.

The hair tress was then subjected to a water rinse using copious amounts of deionized water, followed by a shampoo treatment with embrocation for 30 seconds. The shampoo treatment involved the application of Pantene Pro-V Sheer Volume shampoo (Proctor & Gamble, Cincinnati, Ohio) to the hair as follows. A quarter-sized drop of the shampoo was distributed evenly over the hair tress and then was massaged aggressively into the hair for 30 sec, after which the hair tress was rinsed with water to remove the shampoo. The hair tress was then dried at room temperature. The shampooing step was repeated 9 more times to give a total of 10 shampoo treatments. The entire procedure described above was repeated without the addition of the hair-binding peptide to serve as a control.

The color intensity after the shampoo was measured as described in Examples 4-7. The results are summarized in Table 8.

TABLE 8
Results of Color Retention After Shampoo Treatment

| | | | Peptide | |
|---|---|---|---|---|
| Example | Peptide | SEQ ID NO: | Conc. wt. % | Delta E |
| 8 | HC 71 | 41 | 0.25 | 32.36 |
| 9 | HC 74 | 42 | 0.25 | 33.64 |
| 10 | None | — | 0 | 29.70 |
| Comparative | | | | |

As can be seen from the results in Table 8, the use of either hair-binding peptide (Examples 8 and 9) as a sealant for the dye provided significantly better hair color retention, as measured by the Delta E values, than the use of the dye alone (Comparative Example 10).

### Examples 11-13

### Coloring Hair Using Peptides Having Affinity for Hair as a Sealant

The purpose of these Examples was to demonstrate the coloring of hair using a hair dye in combination with peptide comprising a hair-binding peptide block and charged terminal peptide block as a sealant. The peptides used in this Example were prepared as described in Examples 1 and 2. The color retention was quantified using a spectrophotometric measurement technique.

A peptide given as SEQ ID NO:41 or SEQ ID NO:42 (38 mg), produced via fermentation as described in Examples 1 and 2, was added to 15 g of an aqueous 0.50 wt % solution of Basic Violet 2 (Abbey Color, Philadelphia, Pa.) and the solution was allowed to stir for 1 h. A natural white hair tress (1 cm wide, potted, International Hair Importers & Products Inc., Bellerose, N.Y.) was inserted into a 15 mm×125 mm test tube and 13 mL of the peptide/dye mixture was injected into the test tube. The hair tress was immersed in contact with the colorant solution for 30 min and the colorant solution was agitated using a magnetic stirrer. The tress was removed, allowed to drip dry, and then air dried for 30 min.

The hair tress was then subjected to a water rinse using copious amounts of deionized water, followed by a shampoo treatment with embrocation for 30 seconds. The shampoo treatment involved the application of Pantene Pro-V Sheer Volume shampoo (Proctor & Gamble, Cincinnati, Ohio) to the hair as follows. A quarter-sized drop of the shampoo was distributed evenly over the hair tress and then was massaged aggressively into the hair for 30 sec, after which the hair tress was rinsed with water to remove the shampoo. The hair tress was then dried at room temperature. The shampooing step was repeated 9 more times to give a total of 10 shampoo treatments. The entire procedure described above was repeated without the addition of the hair-binding peptide to serve as a control.

The color intensity after the shampoo was measured as described in Examples 4-7. The results are summarized in Table 9.

TABLE 9
Results of Color Retention After Shampoo Treatment

| | Peptide | | Peptide | |
|---|---|---|---|---|
| Example | ID | SEQ ID NO: | Conc. wt. % | Delta E |
| 11 | HC 71 | 41 | 0.25 | 37.02 |
| 12 | HC 74 | 42 | 0.25 | 38.91 |
| 13 | None | — | 0 | 37.34 |
| Comparative | | | | |

As can be seen from the results in Table 9, the use of peptide given as SEQ ID NO:41 (Example 11) as a sealant for the dye provided no improvement in hair color retention, as measured by the Delta E values, compared to the use of the dye alone (Comparative Example 13). The use of the peptide given as SEQ ID NO:42 (Example 12) as a sealant for the dye, provided a small improvement in hair color retention, as measured by the Delta E values, compared to the use of the dye alone (Comparative Example 13). For the Basic Violet dye it may be necessary to further optimize the ratio of the dye and the peptide to achieve maximum enhancement of color retention.

### Examples 14-16

### Coloring Hair Using Peptides having Affinity for Hair as a Sealant

The purpose of these Examples was to demonstrate the coloring of hair using a hair dye in combination with peptide comprising a hair-binding peptide block and a charged terminal peptide block as a sealant. The peptides used in this Example were prepared as described in Examples 1 and 2. The color retention was quantified using a spectrophotometric measurement technique.

A peptide given as SEQ ID NO:41 or SEQ ID NO:42 (38 mg), produced via fermentation as described in Examples 1 and 2, was added to 15 g of an aqueous 0.50 wt % solution of HC Blue 26 (Aldrich) and the solution was allowed to stir for 1 h. A natural white hair tress (1 cm wide, potted, International Hair Importers & Products Inc., Bellerose, N.Y.) was inserted into a 15 mm×125 mm test tube and 13 mL of the peptide/dye mixture was injected into the test tube. The hair tress was immersed in contact with the colorant solution for 90 min and the colorant solution was agitated using a magnetic stirrer. The tress was removed, allowed to drip dry, and then air dried for 30 min.

The hair tress was then subjected to a water rinse using copious amounts of deionized water, followed by a shampoo treatment with embrocation for 30 seconds. The shampoo treatment involved the application of Pantene Pro-V Sheer Volume shampoo (Proctor & Gamble, Cincinnati, Ohio) to the hair as follows. A quarter-sized drop of the shampoo was distributed evenly over the hair tress and then was massaged aggressively into the hair for 30 sec, after which the hair tress was rinsed with water to remove the shampoo. The hair tress was then dried at room temperature. The shampooing step was repeated 5 more times to give a total of 6 shampoo treatments. The entire procedure described above was repeated without the addition of the hair-binding peptide to serve as a control.

The color intensity after the shampoo was measured as described in Examples 4-7. The results are summarized in Table 10.

TABLE 10
Results of Color Retention After Shampoo Treatment

| | Peptide | | Peptide | |
|---|---|---|---|---|
| Example | ID | SEQ ID NO: | Conc. wt. % | Delta E |
| 14 | HC 71 | 41 | 0.25 | 27.83 |
| 15 | HC 74 | 42 | 0.25 | 30.36 |
| 16 | None | — | 0 | 25.75 |
| Comparative | | | | |

As can be seen from the results in Table 10, the use of either hair-binding peptide (Examples 14 and 15) as a sealant for the dye provided significantly better hair color retention, as measured by the Delta E values, than the use of the dye alone (Comparative Example 16)

### Example 17

### Uptake of Peptides by Hair

The purpose of this Example was to compare the speed of uptake by measuring the amount of various peptides including peptides of the invention bound to hair at different time points using a high performance liquid chromatography (HPLC) method.

The peptides, HC 71, HC 74, IB5 (the hair-binding peptide sequence in HC 71), and A5 (the hair-binding peptide sequence in HC 74), were dissolved in 10 mL of 20 mM Tris, pH 7.2 buffer containing 0.15 M NaCl to give the peptide concentrations shown in Table 11.

Natural white hair, 0.54 g, (International Hair Importers & Products Inc., Bellerose, N.Y.) was pre-equilibrated with 10 mL of Tris buffer for 30 min with mixing at room temperature. Then, the buffer was drained from the hair and 10 mL of one of the peptide solutions was added. The hair was incubated with the peptide solution at room temperature without mixing and samples were taken at 0, 30 and 60 min for HPLC analysis.

For peptide uptake measurements in the presence of Acid Red 33 dye, the peptide solution was mixed 1:1 with an aqueous solution of the dye (0.5 wt %). Natural white hair (0.65 g) pre-wet with deionized water, was added to the peptide solutions and the solutions were incubated at room temperature without mixing. Samples were taken at time 0, 30, and 60 min for analysis by HPLC.

The samples were analyzed using HPLC to determine the concentration of the peptide remaining in solution, and by difference, the amount of peptide taken up by the hair. The HPLC analysis was done using an Agilent liquid chromatograph (Agilent Technologies Inc., Wilmington, Del.) with a 5 μm Zorbax Eclipse XDB-C18 column (Agilent Technologies Inc.). The sample was diluted with distilled water containing 0.1% trifluoroacetic acid (typically a 10-fold dilution), and 5 to 50 μL was injected using a 100 μL injection loop. A solvent gradient elution was used with two solvents: Solvent A, consisting of acetonitrile with 0.1% aqueous trifluoracetic acid, and Solvent B, consisting of water with 0.1% trifluoroacetic acid. The gradient was from 10% acetonitrile to 80% acetonitrile at a rate of 1%/min over a period of 70 min. The flow rate was 1.25 mL/min. Detection was done with a photodiode array detector at 218 nm, 230 nm, and 278 nm. The results are summarized in Table 11.

TABLE 11
Results of HPLC Analysis of Peptide Uptake by Hair

| | | Peptide | % Uptake | % Uptake | % Uptake |
|---|---|---|---|---|---|
| Peptide | SEQ ID | Conc. | time = 0 | time = 30 | time = 60 |
| ID | NO: | mg/mL | min | min | min |
| IB5A | 17 | 0.1 | 0 | 8 | not |
| | | | | | determined |
| HC 71 | 41 | 0.05 | 0 | 69 | 75 |
| A5 | 5 | 0.1 | 0 | 2 | 4 |
| HC 74 | 42 | 0.05 | 0 | 22 | 27 |
| A5/Acid | 5 | 0.1 | 0 | 2 | 8 |
| Red 33 | | | | | |
| HC 74/Acid | 42 | 0.05 | 0 | 13 | 22 |
| Red 33 | | | | | |

As can be seen from the data in Table 11, the peptides having a charged terminal peptide block (peptides HC 71 and HC 74) had a significantly faster uptake onto hair than the same peptides without the charged groups (peptides IB5 and A5, respectively). The faster uptake was also observed in the presence of the Acid Red 33 dye. The faster uptake of the peptides by hair is a significant improvement in terms of practical utility and efficacy.

### Examples 18-27

### Dye Enhancement Using Hair-Binding Peptides Modified with a Charged Terminal Block Co-Applied with a Semi-permanent Dye

A 0.5% aqueous solution of Arianor Ebony (Sensient Technologies Corp, Milwaukee, Wis.) containing 3% benzyl alcohol and 0.05% diazolidinyl urea (biocide) was prepared. Dye insolubles were removed by filtration and the pH of the dye solution was adjusted to 8.50-9.0, using ethanolamine.

Approximately 1 mL of the dye solution was added to selected wells of a 24-well plate (Thomson Instrument Co., Oceanside, Calif.; part # 931565). A multi-block peptide (see Table 12) was dissolved in a solution consisting of 3 wt % benzyl alcohol and 95 wt % water to yield a peptide concentration of 2 wt % or approximately 20 mg/mL. The pH was adjusted to 8.5-9.0 using ethanolamine. The peptide solution was then pipetted into test wells containing dye solution in the amount of 1 mL or 0.2 mL respectively. The total volume in each test well was then adjusted to 2 mL where necessary using 3% aqueous benzyl alcohol (pH adjusted to 8.5-9.0 with ethanolamine). Peptide free controls consisted of 1 mL of dye solution diluted to 2 mL with the pH adjusted aqueous benzyl alcohol solution.

Baseline calorimetric readings were made on one inch long tresses (see below) using an X-RITE® SP78™ Sphere Spectrophotometer (X-Rite, Inc., Grandville, Mich.), by placing the hair tress into the photosensor and calculating L*, a* and b* parameters representing the photometer response. All measurements were the average of three individual determinations.

The tresses (potted on one end with resin) of natural white, 85% gray or regular bleached hair (International Hair Importers and Products, Bellerose, N.Y.) were immersed in the peptide/dye solution and the entire well plate was agitated on a VORTEX-GENIE® 2 Mixer (Scientific Industries, Bohemia, N.Y.) at a speed setting of 1 for 30 minutes. The tresses were removed from the wells, rinsed for 30 seconds under flowing deionized water and then air dried.

The following complement of beads was then added to each well containing a hair tress: (8 beads in all; 4 Novagen Coli Rollers Plating Beads—sterilized (EMD Biosciences, San Diego, Calif.; Catalog No. 71013), 2 BioSpec 6.35 mm glass beads (BioSpec Products, Bartlesville, Okla.; Catalog No. 11079635), and 2 BioSpec 3.2 mm Stainless Steel Bead (Catalog No. 11079132ss).

Then 2.5 mL of aqueous 50% Pantene Pro V® shampoo (Proctor & Gamble Co., Cincinnati, Ohio) was pipetted into each well. The plate was placed on a VWR® VX-2500 Multi-tube Vortexer (VWR, West Chester, Pa.) for 5 minutes to simulate shampooing with embrocation. The hair tresses were then rinsed under flowing deionized water and air dried. This cycle was repeated two more times except that the length of time was 10 minutes for both subsequent embrocation cycles. (This treatment was previously determined to simulate approximately 12 manual shampoo cycles.) At the conclusion of the third embrocation cycle L*, a* and b* values were measured and Delta E values (ΔE) were calculated using equation 1 below:

Delta E=((L*₁−L*₂)²+(a₁−a₂)²+(b₁−b₂)²)^{1/2} (1)

where L*=the lightness variable and a* and b* are the chromaticity coordinates of CIELAB colorspace as defined by the International Commission of Illumination (CIE) (Minolta, Precise Color Communication—Color Control From Feeling to Instrumentation, Minolta Camera Co., 1996).

The Delta E value (ΔE) indicates color retention relative to the non-dyed hair and larger Delta E values indicate better performance. Values listed in the table represent the difference in Delta E values between the peptide-containing sample and the peptide free control. This difference is indicative of color retention after shampoo or water rinse treatments and positive values indicate better performance relative to the peptide free controls (Table 12).

Each of the multi-block peptides are comprised of at least one body surface-binding peptide (hair-binding peptide) domain flanked by a charged peptide block. Examples of the hair-binding peptides (Table 12, bold) include A5 (EQISGSLVAAPW; SEQ ID NO: 5), IB5A (TPPELLHGAPRS; SEQ ID NO: 17), KF11 (NTSQLST; SEQ ID NO: 18), F4 (LDTSFHQVPFHQ; SEQ ID NO: 57), and IB5 (TPPELLHGDPRS; SEQ ID NO: 16).

TABLE 12

Co-Application of a Multi-block Peptide with a Semi-
permanent Dye for Dye Enhancement

| | | | | | |
|---|---|---|---|---|---|
| | | | | | ΔE Retention |
| | | | ΔE Retention | ΔE Retention | Ebony Dye |
| | | SEQ | Ebony dye | Ebony Dye | Regular |
| Example | Peptide ID | ID | with Natural | with 85% | Bleached |
| No. | (formula) | NO. | White Hair | Gray Hair | Hair |

| | | | | | |
|---|---|---|---|---|---|
| 18 | A5-EGEGER | 58 | 5.7 | 3.7 | −1.0 |
| 19 | HC71 | 41 | 5.2 | 2.9 | 1.9 |
| | (P-IB5A-KRKRKD) | | | | |
| 20 | HC74 | 42 | 8.5 | 7.5 | 0.1 |
| | (P-A5-KRKRKD) | | | | |
| 21 | HC81 | 43 | 0.6 | 7.58 | −0.1 |
| | (KF11-GGG- | | | | |
| | GHGHQKQHGLGH | | | | |
| | GHKHGHGHGHGH | | | | |
| | GK) | | | | |
| 22 | HC100 | 59 | −4.5 | 4.1 | −0.27 |
| | (P- | | | | |
| | KRGRHKRPKRHK- | | | | |
| | GGG-IB5A-C) | | | | |
| 23 | F4-KRKRKD | 60 | 0.12 | 2.87 | −2.66 |
| 24 | A5-KRKRKD | 61 | 5.8 | −0.59 | 1.09 |
| 25 | IB5-KRKRKD | 62 | 4.49 | 1.2 | −0.44 |
| 26 | F4 | 57 | 5.17 | 3.24 | 0.95 |
| 27 | KF11-EGEGED | 63 | 3.6 | 0.94 | −4.05 |

Delta E values from the calorimetric readings were judged to show a benefit for color retention when the value was greater than 2 and a substantial benefit when the value was greater than 4. There are many possible mechanisms that may operate but presumably those peptides exhibiting benefit vs. peptide free controls are most effective at bridging the interfacial regions between the hair and the dye molecules in a shampoo resistant fashion.

### Example 28

### Dye Enhancement Using Hair-Binding Peptides Modified with a Charged Terminal Block Applied to Dyed Hair as a Protective Sealant

A 0.5% aqueous Arianor Sienna Brown (Basic Brown 17; Sensient Technologies Corp, Milwaukee, Wis.) solution, containing 3% benzyl alcohol and 0.05% diazolidinyl urea (biocide) was prepared. This dye solution was divided into two equal volumes, one unmodified at pH4 and a second portion adjusted to pH ranged from 8.50 to 9.0, using ethanolamine.

Minitresses (1 inch long) of natural white hair (International Hair Importers and Products, Bellerose, N.Y.) were immersed into the acidic or basic dye solutions for 30 minutes, water rinsed and then air dried overnight. The tresses were then cut into individual sections weighing 30-75 mg each. L*, a*, b* values for individual dyed hair tresses were then measured using a spectrophotometer.

Aqueous peptide solutions varying in concentration were adjusted by addition of ethanolamine to pH 4-5-5.0 or made slightly basic (pH 8.5-9.0). The 3% benzyl alcohol diluent was also pH adjusted in the same fashion.

Each cavity of a well plate (Thomson Instrument Co. part no. 931565, 24-10 ml wells), contained the assortment of beads (8 beads in all; 4 Novagen Coli Rollers Plating Beads—sterilized (EMD Biosciences, San Diego, Calif.; Catalog No. 71013), 2 BioSpec 6.35 mm glass beads (BioSpec Products, Bartlesville, Okla.; Catalog No. 11079635), and 2 BioSpec 3.2 mm Stainless Steel Bead (Catalog No. 11079132ss).

Target peptide and diluent were added using an automatic pipette. Controls without peptide were made using pH-adjusted diluent only. After several minutes of mixing (VORTEX-GENIE® 2, Scientific Industries, Bohemia, N.Y.) a small dyed hair tress was added to each well and stirred for 30 minutes on the vortex mixer. When stirring was complete, the hair tresses were removed from the wells and rinsed under running deionized water for 20-30 seconds.

The wet hair tresses were inserted into a modified well plate into which holes had been drilled to allow them to be air dried. Once the tresses were dried, colorimetric readings were taken for color loss after water rinse. Hair tresses were reinserted into their respective wells along with beads and 2.5 mL of aqueous 50% Pantene Pro V® shampoo was pipetted onto the hair. The plate was placed into a VWR® VX-2500 Multi-tube Vortexer (VWR, West Chester, Pa.) for 5-15 minutes to simulate shampooing with embrocation. The hair tresses were then rinsed under flowing deionized water and air dried. This cycle was repeated according to the number of simulated embrocation cycles desired. Colorimetric data for color loss was compiled and plotted against peptide free controls. A difference of at least 3 Delta E units verses controls was judged to be sufficient for differentiating peptide performance verses peptide free controls.

KF11 (NTSQLST; SEQ ID NO: 18) is a shampoo-resistant hair-binding domain (Table 13, bold). A dyed-hair-binding peptide is provided as Dye-1 (see co-pending U.S. Provisional Patent Application No. 60/972,312.) PMMA4A (HTHHDTHKPWPTDAHRNSSV; SEQ ID NO: 64) and PMMA9 (IDTFYMSTMSHS; SEQ ID NO: 65) are binding domains having a high affinity for polymethylmethacrylate (Table 13, bold).

TABLE 13

Application of a Charged Multi-block Peptide for
Dye Enhancement

| | | | | | | |
|---|---|---|---|---|---|---|
| Peptide | Sequence/formula | Shampoo | | ΔE | | ΔE |
| ID | (SEQ ID NO:) | Equivalents | pH | Loss | pH | Loss |

| | | | | | | |
|---|---|---|---|---|---|---|
| Dye-1 | SSNYNYNYNYQYSSR | 12 | — | — | 3.9 | 14 |
| | (SEQ ID NO: 66) | | | | | |
| Dye-1 | SSNYNYNYNYQYSSR- | 25 | 8.7 | 3.22 | 4.5 | 6 |
| Cationic | KRKRKD | | | | | |
| | (SEQ ID NO: 67) | | | | | |
| Dye-1 | SSNYNYNYNYQYSSR- | 12 | 8.8 | 0.34 | 4.5 | 1.93 |
| Anionic | EGEGER | | | | | |
| | (SEQ ID NO: 68) | | | | | |
| HC130 | PG-PMMA4A-GAG- | 12 | 8.8 | 6.95 | 5.1 | 11.56 |
| | PMMA4A- | | | | | |
| | GGSGPGSGG-KF11- | | | | | |
| | GGG-KF11-GGPKK | | | | | |
| | (SEQ ID NO: 69) | | | | | |
| HC133 | PG-PMMA9-GAG- | 12 | — | — | 5.7 | 11.41 |
| | PMMA9- | | | | | |
| | GGSGPGSGG-KF11- | | | | | |
| | GS(KGGGS)4-KF11- | | | | | |
| | GGPWKK | | | | | |
| | (SEQ ID NO: 70) | | | | | |

### Example 29

### Prophetic

### Selection of Tooth-Binding Peptides Using Biopanning

The purpose of this prophetic Example is to describe how to identify phage peptides that bind to teeth with high affinity.

Extracted human teeth, which may be obtained from a dental office, are cleaned by brushing with soap solution, rinsed with deionized water, and allowed to air-dry at room temperature. The teeth are placed in a 15 mL centrifuge tube (Corning Inc., Acton, Mass.), one tooth per tube. The teeth samples are treated for 1 h with blocking buffer consisting of 1 mg/mL BSA in TBST-0.5%, and then washed with TBST-0.5%. The teeth samples are incubated with the phage library (Ph.D-12 Phage Display Peptide Library) for 15 to 30 min at room temperature and then washed 10 times using TBST-0.5%.

The teeth are then transferred to clean tubes and non-specific acidic elution buffer consisting of 1 mg/mL BSA in 0.2 M glycine-HCl, pH 2.2, is added to each tube and incubated for 10 min to elute the bound phages. Then, the teeth samples are washed three times with the elution buffer and washed three times with TBST-0.5%. The acid-treated teeth, which have acid resistant phage peptides still attached, are used to directly infect 500 μL of mid-log phase bacterial host cells, E. coli ER2738 (New England BioLabs), which are then grown in LB medium for 20 min and then mixed with 3 mL of agarose top (LB medium with 5 mM MgCl₂, and 0.7% agarose) at 45° C. This mixture is spread onto a LB medium/IPTG/S-Gal™ plate (LB medium with 15 g/L agar, 0.05 g/L IPTG (isopropyl β-D-thiogalactoside), and 0.04 g/L S-Gal™ (isopropyl β-D-thiogalactoside)) and is incubated overnight at 37° C. The black plaques are counted to calculate the phage titer. The single black plaques are randomly picked for DNA isolation and sequencing analysis.

The amplified and isolated phages are contacted with a fresh tooth sample and the biopanning procedure is repeated two more times. After the third round of biopanning, the acid-treated teeth are used to directly infect E. coli ER2738 cells, and the cells are cultured as described above. Single black plaques are randomly picked for DNA isolation and sequence analysis. The single plaque lysates are prepared following the manufacture's instructions (New England BioLabs) and the single stranded phage genomic DNA is purified using the QIAprep Spin M13 Kit (Qiagen, Valencia, Calif.) and sequenced. The identified peptide sequences will have a binding affinity for teeth.

### Example 30

### Selection of Tooth Pellicle Binding Peptides Using Standard Biopanning

The purpose of this Example was to identify phage peptides that bind tooth pellicle using standard phage display biopanning.

The compressed HAP disks (Hydroxyapatite disk, 3 mm diameter) were used to form the pellicles by incubating the disks inside a human mouth for 1.5 hours followed by TBS rinse. The disks were then incubated in SUPERBLOCK™ blocking buffer (Pierce Chemical Company, Rockford, Ill.; Prod. #37535) for 1 hour at room temperature, followed by 3 washes with TBST (TBS in 0.05% TWEEN™ 20). Libraries of phage containing random peptide inserts (10¹¹ pfu) from 7 to 20 amino acids were added to each tube. After 60 minutes of incubation at room temperature and shaking at 50 rpm, unbound phage were removed by aspirating the liquid out of each well followed by 6 washes with 1.0 mL TBS containing the detergent TWEEN® 20 (TBST) at a final concentration of 0.05%.

The sample disks were then transferred to clean tubes and 200 μL of elution buffer consisting of 1 mg/mL BSA in 0.2 M glycine-HCl, pH 2.2, was added to each well and incubated for 10 min to elute the bound phages. Then, 32 μL of neutralization buffer consisting of 1 M Tris-HCl, pH 9.2, was added to each well. The phage particles, which were in the elution buffer as well as on the sample disks, were amplified by incubating with diluted E. coli ER2738 cells, from an overnight culture diluted 1:100 in LB medium, at 37° C. for 4.5 h. After this time, the cell culture was centrifuged for 30 s and the upper 80% of the supernatant was transferred to a fresh tube, ⅙ volume of PEG/NaCl (20% polyethylene glycol-800, 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This was the first round of amplified stock. The amplified first round phage stock was then titered according to the standard protocol. For the 2^{nd}, 3^{rd} and 4^{th} round of biopanning, more than 2×10¹¹ pfu of phage stock from the previous round was used. The biopanning process was repeated for 2 more rounds under the same conditions as described above. The same biopanning condition was used for the 4^{th} round, except the washing solution was TBS with 0.5% TWEEN™ 20 instead of 0.05% TWEEN™ 20.

After the 4^{th} round of biopanning, 95 random single phage plaque lysates were prepared following the manufacture's instructions (New England Biolabs) and the single stranded phage genomic DNA was purified using the QIAprep Spin M13 Kit (Qiagen, Valencia, Calif.) and sequenced at the DuPont Sequencing Facility using −96 gIII sequencing primer (5′-CCCTCATAGTTAGCGTAACG-3′), given as SEQ ID NO: 71. The displayed peptide is located immediately after the signal peptide of gene III. Based on the peptide sequences, 20 phage candidates were selected for further pellicle binding analysis.

### Example 31

### Characterization of Tooth Pellicle Binding Candidates on Pellicle Surface

A total of 20 selected phage candidates (Example 29) were used in a phage ELISA experiment. Purified phage lysates were used for binding to pellicle HAP disks using an anti-M13 phage antibody conjugated to horseradish-peroxidase, followed by the addition of chromogenic agent TMB, obtained from Pierce Biotechnology (Rockford, Ill.). The plates were read at A₄₅₀ nm.

For each phage candidate to be tested, the pellicle coated HAP disks (3 mm diameter) was incubated for 1 h at room temperature with 200 μL of blocking buffer, consisting of 2% non-fat dry milk (Schleicher & Schuell, Inc.) in TBS. The blocking buffer was removed by aspirating the liquid out of each tube. The tube was rinsed 6 times with wash buffer consisting of TBST-0.05%. The wells were filled with 200 μL of TBST-0.5% containing 1 mg/mL BSA (bovine serum albumin) and then 10 μL (over 10¹⁰ pfu) of purified phage stock was added. The samples were incubated at room temperature for 60 min with slow shaking. The non-binding phage were removed by washing 6 times with TBST-0.5%. Then, 100 μL of horseradish peroxidase/anti-M13 antibody conjugate (Amersham USA, Piscataway, N.J.), diluted 1:500 in the blocking buffer, was added and incubated for 1 h at room temperature. The conjugate solution was removed and was washed 6 times with TBST-0.5%. TMB (3,3′,5,5′-tetramethylbenzidine) substrate (200 μL), obtained from Pierce Biotechnology (Rockford, Ill.) was added to each well and the color was allowed to develop for 5 to 30 min, typically for 10 min, at room temperature. Then, stop solution (200 μL of 2 M H₂SO₄) was added to each well and the solution was transferred to a 96-well plate and the A₄₅₀ was measured using a microplate spectrophotometer (Molecular Devices, Sunnyvale, Calif.). The resulting absorbance values are given in Table 14.

TABLE 14

Amino Acid Sequences of Pellicle Binding
Peptides

| | | | |
|---|---|---|---|
| | | | O.D. |
| | | SEQ | at |
| Phage | | ID | 450 |
| ID | Amino Acids Sequence | NO. | nm |

| | | | |
|---|---|---|---|
| Control | No phage | — | 0.218 |
| Pell 1 | AHPESLGIKYALDGNSDPHA | 72 | 0.739 |
| Pell 2 | ASVSNYPPIHHLATSNTTVN | 73 | 0.75 |
| Pell 3 | DECMEPLNAAHCWR | 74 | 0.49 |
| Pell 4 | DECMHGSDVEFCTS | 75 | 0.664 |
| Pell 5 | DLCSMQMMNTGCHY | 76 | 0.83 |
| Pell 6 | DLCSSPSTWGSCIR | 77 | 0.735 |
| Pell 7 | DPNESNYENATTVSQPTRHL | 78 | 0.831 |
| Pell 8 | EPTHPTMRAQMHQSLRSSSP | 79 | 0.712 |
| Pell 9 | GNTDTTPPNAVMEPTVQHKW | 80 | 0.755 |
| Pell 10 | NGPDMVQSVGKHKNS | 81 | 0.729 |
| Pell 11 | NGPEVRQIPANFEKL | 82 | 0.607 |
| Pell 12 | NNTSADNPPETDSKHHLSMS | 83 | 0.521 |
| Pell 13 | NNTWPEGAGHTMPSTNIRQA | 84 | 0.598 |
| Pell 14 | NPTATPHMKDPMHSNAHSSA | 85 | 0.7 |
| Pell 15 | NPTDHIPANSTNSRVSKGNT | 86 | 0.567 |
| Pell 16 | NPTDSTHMMHARNHE | 87 | 0.578 |
| Pell 17 | QHCITERLHPPCTK | 88 | 0.614 |
| Pell 18 | TPCAPASFNPHCSR | 89 | 0.416 |
| Pell 19 | TPCATYPHFSGCRA | 90 | 0.731 |
| Pell 20 | WCTDFCTRSTPTSTSRSTTS | 91 | 0.715 |

### Example 32

### Selection of Tooth Enamel Binding Peptides Using Standard Biopanning

The purpose of this example was to identify phage peptides that bind tooth enamel using standard phage display biopanning.

The unpolished bovine enamel blocks from incisors (3 mm squares) and polished bovine enamel disks from the incisors (3 mm diameter disks) were embedded in wax to form a well with only the intended surfaces exposed. The enamel surfaces were then incubated in SUPERBLOCK® blocking buffer (Pierce Chemical Company, Rockford, Ill.; Prod. #37535) for 1 hour at room temperature, followed by 3 washes with TBST (TBS in 0.05% TWEEN® 20). Libraries of phage containing random peptide inserts (10¹¹ pfu) from 7 to 20 amino acids were added to the enamel well for 10 minutes pre-absorption to titrate the wax surface, unbound phage were removed by aspirating the liquid out of each well. Then, 100 μL of the same phage library (10¹¹ pfu) was added to the enamel well for 60 min incubation at room temperature with slow 50 rpm shaking, followed by 6 washes with 1.0 mL TBS containing the detergent TWEEN® 20 (TBST) at a final concentration of 0.05%.

The enamel blocks (or polished disks) were then cut out of the wax well and transferred to a clean tube and 200 μL of elution buffer consisting of 1 mg/mL BSA in 0.2 M glycine-HCl, pH 2.2, was added to each well and incubated for 10 min to elute the bound phages. Then, 32 μL of neutralization buffer consisting of 1 M Tris-HCl, pH 9.2, was added to each tube. The phage particles, which were in the elution buffer as well as on the enamel blocks, were amplified by incubating with diluted E. coli ER2738 cells, from an overnight culture diluted 1:100 in LB medium, at 37° C. for 4.5 h. After this time, the cell culture was centrifuged for 30 s and the upper 80% of the supernatant was transferred to a fresh tube, ⅙ volume of PEG/NaCl (20% polyethylene glycol-800, 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This was the first round of amplified stock. The amplified first round phage stock was then titered according to the standard protocol. For the 2^{nd} round of biopanning, more than 2×10¹¹ pfu of phage stock from the previous round was used. The biopanning process was repeated for 1 more rounds under the same conditions as described above. The same biopanning condition was used for the 3^{rd} round, except the washing solution was TBS with 0.5% TWEEN® 20 instead of 0.05% TWEEN® 20.

After the 3^{rd} round of biopanning, 95 random single phage plaque lysates were prepared following the manufacture's instructions (New England Biolabs) and the single stranded phage genomic DNA was purified using the QIAprep Spin M13 Kit (Qiagen, Valencia, Calif.) and sequenced at the DuPont Sequencing Facility using −96 gill sequencing primer (5′-CCCTCATAGTTAGCGTAACG-3′), given as SEQ ID NO: 71. The displayed peptide is located immediately after the signal peptide of gene III. Based on the peptide sequences, 20 phage candidates were selected for further pellicle binding analysis (Table 15). “BoEn” means bovine enamel and “BoEn P” means polished bovine enamel.

TABLE 15
Bovine Enamel Binding Peptide Sequences

| | | |
|---|---|---|
| | | SEQ |
| | | ID |
| Phage ID | Amino Acid Sequence | NO: |

| | | |
|---|---|---|
| BoEn P1 | APPLKTYMQERELTMSQNKD | 92 |
| BoEn P2 | EPPTRTRVNNHTVTVQAQQH | 93 |
| BoEn P3 | GYCLRGDEPAVCSG | 94 |
| BoEn P4 | LSSKDFGVTNTDQRTYDYTT | 95 |
| BoEn P5 | NFCETQLDLSVCTV | 96 |
| BoEn P6 | NTCQPTKNATPCSA | 97 |
| BoEn P7 | PSEPERRDRNIAANAGRFNT | 98 |
| BoEn P8 | THNMSHFPPSGHPKRTAT | 99 |
| BoEn P9 | TTCPTMGTYHVCWL | 100 |
| BoEn P10 | YCADHTPDPANPNKICGYSH | 101 |
| BoEn 1 | AANPHTEWDRDAFQLAMPPK | 102 |
| BoEn 2 | DLHPMDPSNKRPDNPSDLHT | 103 |
| BoEn 3 | ESCVSNALMNQCIY | 104 |
| BoEn 4 | HNKADSWDPDLPPHAGMSLG | 105 |
| BoEn 5 | LNDQRKPGPPTMPTHSPAVG | 106 |
| BoEn 6 | NTCATSPNSYTCSN | 107 |
| BoEn 7 | SDCTAGLVPPLCAT | 108 |
| BoEn 8 | TIESSQHSRTHQQNYGSTKT | 109 |
| BoEn 9 | VGTMKQHPTTTQPPRVSATN | 110 |
| BoEn 10 | YSETPNDQKPNPHYKVSGTK | 111 |

### Example 33

### Characterization of Tooth Enamel Binding Peptide Candidates on Enamel Surface

A total of 11 selected phage candidates (Example 32) was used in a phage ELISA experiment. Purified phage lysates were used for binding to the enamel blocks using an anti-M13 phage antibody conjugated to horseradish-peroxidase, followed by the addition of chromogenic agent TMB, obtained from Pierce Biotechnology (Rockford, Ill.). The plates were read at A450 nm.

For each phage candidate to be tested, the polished and unpolished enamel blocks were incubated for 1 h at room temperature with 200 μL of blocking buffer, consisting of 2% non-fat dry milk (Schleicher & Schuell, Inc.) in TBS. The blocking buffer was removed by aspirating the liquid out of each tube. The tube was rinsed 6 times with wash buffer consisting of TBST-0.05%. The wells were filled with 200 μL of TBST-0.5% containing 1 mg/mL BSA and then 10 μL (over 10¹⁰ pfu) of purified phage stock was added. The samples were incubated at room temperature for 60 min with slow shaking. The non-binding phage was removed by washing 6 times with TBST-0.5%. Then, 100 μL of horseradish peroxidase/anti-M13 antibody conjugate (Amersham USA, Piscataway, N.J.), diluted 1:500 in the blocking buffer, was added and incubated for 1 h at room temperature. The conjugate solution was removed and was washed 6 times with TBST-0.5%. TMB substrate (200 μL), obtained from Pierce Biotechnology (Rockford, Ill.) was added to each well and the color was allowed to develop for 5 to 30 min, typically for 10 min, at room temperature. Then, stop solution (200 μL of 2 M H₂SO₄) was added to each well and the solution was transferred to a 96-well plate and the A₄₅₀ was measured using a microplate spectrophotometer (Molecular Devices, Sunnyvale, Calif.). The resulting absorbance values,) are given in Tables 16 and 17. BoEn means bovine enamel and BoEn P means polished bovine enamel.

TABLE 16

Phage ELISA Results on Bovine Enamel Binding
Assay of Selected Phage Candidates

| | | | |
|---|---|---|---|
| | | | O.D. |
| | | SEQ | at |
| Phage | | ID | 450 |
| ID | Amino Acid Sequence | NO: | nm |

| | | | |
|---|---|---|---|
| Control | no phage | — | 0.112 |
| BoEn P2 | EPPTRTRVNNHTVTVQAQQH | 93 | 0.641 |
| BoEn P3 | GYCLRGDEPAVCSG | 94 | 0.665 |
| BoEn P5 | NFCETQLDLSVCTV | 96 | 0.797 |
| BoEn P6 | NTCQPTKNATPCSA | 97 | 0.83 |
| BoEn P8 | THNMSHFPPSGHPKRTAT | 99 | 2.02 |

TABLE 17

Phage ELISA Results on Bovine Polished Enamel
Binding Assay of Selected Phage Candidates

| | | | |
|---|---|---|---|
| | | | O.D. |
| | | SEQ | at |
| Phage | | ID | 450 |
| ID | Amino Acid Sequence | NO: | nm |

| | | | |
|---|---|---|---|
| Control | no phage | — | 0.193 |
| BoEn 1 | AANPHTEWDRDAFQLAMPPK | 102 | 1.402 |
| BoEn 5 | LNDQRKPGPPTMPTHSPAVG | 106 | 0.944 |
| BoEn 6 | NTCATSPNSYTCSN | 107 | 2.38 |
| BoEn 7 | SDCTAGLVPPLCAT | 108 | 0.892 |
| BoEn 9 | VGTMKQHPTTTQPPRVSATN | 110 | 0.568 |
| BoEn 10 | YSETPNDQKPNPHYKVSGTK | 111 | 3.942 |

What is claimed is:

1. A peptide having affinity for a body surface having the general structure:
(nCPB^{±})ₓ-(Sn)-BSBPB-(Sc)-(cCPB^{±})_{y}
wherein:
(i) nCPB^{±} is an N-terminal charged peptide block, said N-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(ii) cCPB^{±} is a C-terminal charged peptide block, said C-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(iii) BSBPB is a body surface-binding peptide block comprising at least one body surface-binding peptide;
(iv) x and y are independently 0 or 1, provided that x and x may not both be 0; and
(v) Sn and Sc are optional peptide spacers comprised of 0 to about 20 amino acids.

2. A peptide-based body surface reagent comprising a peptide having affinity for a body surface coupled to benefit agent, said peptide-based body surface reagent having the general structure:
{(nCPB^{±})ₓ-(Sn)-BSBPB-(Sc)-(cCPB^{±})_{y}}z-(So-BAₛ)ᵣ,
wherein:
(i) nCPB^{±} is an N-terminal charged peptide block, said N-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(ii) cCPB^{±} is a C-terminal charged peptide block, said C-terminal charged peptide block comprising at least 30 mole % of charged amino acids selected from the group consisting of lysine, arginine, histidine, aspartic acid, glutamic acid, and combinations thereof, and said peptide block being from 1 to about 50 amino acids in length;
(iii) BSBPB is a body surface-binding peptide block comprising at least one body surface-binding peptide;
(iv) BA is a benefit agent;
(v) x and y are independently 0 or 1, provided that x and x may not both be 0;
(vi) z=1 to about 10,000;
(vii) r and s are independently 1 to about 100;
(viii) So is an optional organic spacer; and
(ix) Sn and Sc are optional peptide spacers comprised of 0 to about 20 amino acids.

3. The peptide having affinity for a body surface according to claim 1 or the peptide-based body surface reagent according to claim 2 wherein the N-terminal charged peptide block and the C-terminal charged peptide block comprise at least 50 mole % of charged amino acids selected from the group consisting of lysine, arginine, histidine, aspartic acid, glutamic acid, and combinations thereof.

4. The peptide-based body surface reagent according to claim 2 wherein the organic spacer is selected from the group consisting of ethanolamine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, ethyl alkyl chains, propyl alkyl chains, hexyl alkyl chains, steryl alkyl chains, cetyl alkyl chains, and palmitoyl alkyl chains.

5. The peptide having affinity for a body surface according to claim 1 or the peptide-based body surface reagent according to claim 2 wherein the peptide having affinity for a body surface has affinity for a body surface selected from the group consisting of hair, nails, teeth, gums, skin, and tissues of the oral cavity.

6. The peptide having affinity for a body surface according to claim 1 or the peptide-based body surface reagent according to claim 2 wherein the body surface-binding peptide block comprises at least one body surface-binding peptide which is isolated by a process comprising the steps of:
(i) providing a library of combinatorially generated phage-peptides;
(ii) contacting the library of (i) with a body surface to form a reaction solution comprising:
(A) phage-peptide-body surface complex;
(B) unbound body surface, and
(C) uncomplexed peptides;
(iii) isolating the phage-peptide-body surface complex of (ii);
(iv) eluting the weakly bound peptides from the isolated peptide complex of (iii); and
(v) identifying the remaining bound phage-peptides either by using the polymerase chain reaction directly with the phage-peptide-body surface complex remaining after step (iv), or by infecting bacterial host cells directly with the phage-peptide-body surface complex remaining after step (iv), growing the infected cells in a suitable growth medium, and isolating and identifying the phage-peptides from the grown cells.

7. The peptide having affinity for a body surface according to claim 1 or the peptide-based body surface reagent according to claim 2 wherein the peptide having affinity for a body surface further comprises a proline residue on the N-terminal end and optionally an aspartic acid residue on the C-terminal end.

8. The peptide-based body surface reagent according to claim 2 wherein the benefit agent is selected from the group consisting of colorants, conditioning agents, sunscreen agents, and oral benefit agents.

9. A method for applying a benefit agent to a body surface comprising the steps of:
a) providing a composition comprising a peptide-based body surface reagent according to claim 2; and
b) applying the composition to the body surface for a time sufficient for the peptide-based body surface reagent to bind to the body surface.

10. A method for applying a benefit agent to a body surface comprising the steps of:
a) providing a benefit agent having affinity to the body surface;
b) providing a composition comprising a peptide having affinity for a body surface according to claim 1; and
c) applying the benefit agent and said composition to the body surface for a time sufficient for the benefit agent and the peptide having affinity for the body surface or the peptide based body surface reagent to bind to the body surface.

11. The method according to claim 10 wherein the benefit agent and the composition of (b) are applied to the hair concomitantly.

12. The method according to claim 10 wherein the benefit agent is applied to the body surface prior to the application of the composition of (b).

13. The method according to claim 10 wherein the composition of (b) is applied to the body surface prior to the application of the benefit agent.

14. The method according to claim 10 wherein the benefit agent of step (a) is provided in a form of the peptide-based body surface reagent according to claim 2.

15. The method according to claim 10 further comprising the step of:
d) reapplying the composition of (b) to the body surface for a time sufficient for the peptide having affinity for the body surface or the peptide-based body surface reagent to bind to the body surface.

16. The method according to claim 10 further comprising the step of:
d) applying a composition comprising a polymeric sealant to the hair.

17. The method according to claim 16 wherein the polymeric sealant is selected from the group consisting of poly(allylamine), polyacrylates, acrylate copolymers, polyurethanes, carbomers, methicones, amodimethicones, polyethylenene glycol, beeswax, and siloxanes.

18. A personal care composition comprising the peptide according to claim 1 or the peptide-based body surface reagent according to claim 2.
